SEQUENCE LISTING (48 sequences)

SEQ ID NO: 1 - DNA, 21 bp, synthetic construct
  misc_feature 1..21: note = ASPA-Exon1 forward primer
  source 1..21: mol_type = other DNA
ctccactcaagggaattctgt

SEQ ID NO: 2 - DNA, 20 bp, synthetic construct
  misc_feature 1..20: note = ASPA-Exon1 reverse primer
  source 1..20: mol_type = other DNA
actgcatgtacggacatgaa

SEQ ID NO: 3 - DNA, 19 bp, synthetic construct
  misc_feature 1..19: note = ASPA-Exon2 forward primer
  source 1..19: mol_type = other DNA
agatttggcgactggttct

SEQ ID NO: 4 - DNA, 20 bp, synthetic construct
  misc_feature 1..20: note = ASPA-Exon2 reverse primer
  source 1..20: mol_type = other DNA
tgcaccttccctcataactg

SEQ ID NO: 5 - DNA, 20 bp, synthetic construct
  misc_feature 1..20: note = ASPA-Exon3 forward primer
  source 1..20: mol_type = other DNA
actctgttgaagcaaagaga

SEQ ID NO: 6 - DNA, 20 bp, synthetic construct
  misc_feature 1..20: note = ASPA-Exon3 reverse primer
  source 1..20: mol_type = other DNA
cagagcaagactctgtctca

SEQ ID NO: 7 - DNA, 20 bp, synthetic construct
  misc_feature 1..20: note = ASPA-Exon4 forward primer
  source 1..20: mol_type = other DNA
ttccatgatgctacatggtt

SEQ ID NO: 8 - DNA, 21 bp, synthetic construct
  misc_feature 1..21: note = ASPA-Exon4 reverse primer
  source 1..21: mol_type = other DNA
gcaaatctgacccaggttcca

SEQ ID NO: 9 - DNA, 20 bp, synthetic construct
  misc_feature 1..20: note = ASPA-Exon5 forward primer
  source 1..20: mol_type = other DNA
tgttctcgaactcctgacct

SEQ ID NO: 10 - DNA, 20 bp, synthetic construct
  misc_feature 1..20: note = ASPA-Exon5 reverse primer
  source 1..20: mol_type = other DNA
gcgaagtgctgtatgagcta

SEQ ID NO: 11 - DNA, 19 bp, synthetic construct
  misc_feature 1..19: note = ASPA-Exon6 forward primer
  source 1..19: mol_type = other DNA
gatcaagactggaaaccac

SEQ ID NO: 12 - DNA, 20 bp, synthetic construct
  misc_feature 1..20: note = ASPA-Exon6 reverse primer
  source 1..20: mol_type = other DNA
gaagtgtagtaaggcaaagc

SEQ ID NO: 13 - DNA, 20 bp, synthetic construct
  misc_feature 1..20: note = Endo-OCT4 forward primer
  source 1..20: mol_type = other DNA
cctcacttcactgcactgta

SEQ ID NO: 14 - DNA, 20 bp, synthetic construct
  misc_feature 1..20: note = Endo-OCT4 reverse primer
  source 1..20: mol_type = other DNA
caggttttctttccctagct

SEQ ID NO: 15 - DNA, 19 bp, synthetic construct
  misc_feature 1..19: note = Endo-SOX2 forward primer
  source 1..19: mol_type = other DNA
cccagcagacttcacatgt

SEQ ID NO: 16 - DNA, 20 bp, synthetic construct
  misc_feature 1..20: note = Endo-SOX2 reverse primer
  source 1..20: mol_type = other DNA
cctcccatttccctcgtttt

SEQ ID NO: 17 - DNA, 21 bp, synthetic construct
  misc_feature 1..21: note = Endo-NANOG forward primer
  source 1..21: mol_type = other DNA
gaatcttcacctatgcctgtg

SEQ ID NO: 18 - DNA, 21 bp, synthetic construct
  misc_feature 1..21: note = Endo-NANOG reverse primer
  source 1..21: mol_type = other DNA
atcattgagtacacacagctg

SEQ ID NO: 19 - DNA, 20 bp, synthetic construct
  misc_feature 1..20: note = Exo- OCT4 forward primer
  source 1..20: mol_type = other DNA
cctcacttcactgcactgta

SEQ ID NO: 20 - DNA, 24 bp, synthetic construct
  misc_feature 1..24: note = Exo- OCT4 reverse primer
  source 1..24: mol_type = other DNA
ttatcgtcgaccactgtgctgctg

SEQ ID NO: 21 - DNA, 19 bp, synthetic construct
  misc_feature 1..19: note = Exo- SOX2 forward primer
  source 1..19: mol_type = other DNA
cccagcagacttcacatgt

SEQ ID NO: 22 - DNA, 24 bp, synthetic construct
  misc_feature 1..24: note = Exo- SOX2 reverse primer
  source 1..24: mol_type = other DNA
ttatcgtcgaccactgtgctgctg

SEQ ID NO: 23 - DNA, 20 bp, synthetic construct
  misc_feature 1..20: note = Exo-KLF4 forward primer
  source 1..20: mol_type = other DNA
gatgaactgaccaggcacta

SEQ ID NO: 24 - DNA, 24 bp, synthetic construct
  misc_feature 1..24: note = Exo-KLF4 reverse primer
  source 1..24: mol_type = other DNA
ttatcgtcgaccactgtgctgctg

SEQ ID NO: 25 - DNA, 20 bp, synthetic construct
  misc_feature 1..20: note = Exo-cMYC forward primer
  source 1..20: mol_type = other DNA
gccacagcatacatcctgtc

SEQ ID NO: 26 - DNA, 24 bp, synthetic construct
  misc_feature 1..24: note = Exo-cMYC reverse primer
  source 1..24: mol_type = other DNA
ttatcgtcgaccactgtgctgctg

SEQ ID NO: 27 - DNA, 21 bp, synthetic construct
  misc_feature 1..21: note = Episomal-Exo-OCT4 forward primer
  source 1..21: mol_type = other DNA
ctctagagcctctgctaacca

SEQ ID NO: 28 - DNA, 21 bp, synthetic construct
  misc_feature 1..21: note = Episomal-Exo-OCT4 reverse primer
  source 1..21: mol_type = other DNA
tgtgcatagtcgctgcttgat

SEQ ID NO: 29 - DNA, 21 bp, synthetic construct
  misc_feature 1..21: note = Episomal-Exo-KLF4 forward primer
  source 1..21: mol_type = other DNA
gctcccatctttctccacgtt

SEQ ID NO: 30 - DNA, 22 bp, synthetic construct
  misc_feature 1..22: note = Episomal-Exo-KLF4 reverse primer
  source 1..22: mol_type = other DNA
gaagcttgaattcctgcaggca

SEQ ID NO: 31 - DNA, 21 bp, synthetic construct
  misc_feature 1..21: note = Episomal-Exo-LIN28 forward primer
  source 1..21: mol_type = other DNA
agagcatcagccatatggtag

SEQ ID NO: 32 - DNA, 22 bp, synthetic construct
  misc_feature 1..22: note = Episomal-Exo-LIN28 reverse primer
  source 1..22: mol_type = other DNA
gaagcttgaattcctgcaggca

SEQ ID NO: 33 - DNA, 21 bp, synthetic construct
  misc_feature 1..21: note = Episomal-Exo-L-MYC forward primer
  source 1..21: mol_type = other DNA
ctctagagcctctgctaacca

SEQ ID NO: 34 - DNA, 21 bp, synthetic construct
  misc_feature 1..21: note = Episomal-Exo-L-MYC reverse primer
  source 1..21: mol_type = other DNA
tcgaatttcttccagatgtcc

SEQ ID NO: 35 - DNA, 23 bp, synthetic construct
  misc_feature 1..23: note = ASPA forward primer
  source 1..23: mol_type = other DNA
cggaattcatgacttcttgtcac

SEQ ID NO: 36 - DNA, 24 bp, synthetic construct
  misc_feature 1..24: note = ASPA reverse primer
  source 1..24: mol_type = other DNA
ggactagtctaatgtaaacagcag

SEQ ID NO: 37 - DNA, 19 bp, synthetic construct
  misc_feature 1..19: note = hASPA forward primer
  source 1..19: mol_type = other DNA
gatcaagactggaaaccac

SEQ ID NO: 38 - DNA, 20 bp, synthetic construct
  misc_feature 1..20: note = hASPA reverse primer
  source 1..20: mol_type = other DNA
gcggcctcattcacaaacac

SEQ ID NO: 39 - DNA, 20 bp, synthetic construct
  misc_feature 1..20: note = hMBP forward primer
  source 1..20: mol_type = other DNA
ctataaatcggctcacaagg

SEQ ID NO: 40 - DNA, 20 bp, synthetic construct
  misc_feature 1..20: note = hMBP reverse primer
  source 1..20: mol_type = other DNA
aggcggttatattaagaagc

SEQ ID NO: 41 - DNA, 21 bp, synthetic construct
  misc_feature 1..21: note = mPLP forward primer
  source 1..21: mol_type = other DNA
cacttacaacttcgccgtcct

SEQ ID NO: 42 - DNA, 23 bp, synthetic construct
  misc_feature 1..23: note = mPLP reverse primer
  source 1..23: mol_type = other DNA
gggagtttctatgggagctcaga

SEQ ID NO: 43 - DNA, 19 bp, synthetic construct
  misc_feature 1..19: note = hOLIG2 forward primer
  source 1..19: mol_type = other DNA
tgcgcaagctttccaagat

SEQ ID NO: 44 - DNA, 20 bp, synthetic construct
  misc_feature 1..20: note = hOLIG2 reverse primer
  source 1..20: mol_type = other DNA
cagcgagttggtgagcatga

SEQ ID NO: 45 - DNA, 24 bp, synthetic construct
  misc_feature 1..24: note = hNKX2.2 forward primer
  source 1..24: mol_type = other DNA
gacaactggtggcagatttcgctt

SEQ ID NO: 46 - DNA, 24 bp, synthetic construct
  misc_feature 1..24: note = hNKX2.2 reverse primer
  source 1..24: mol_type = other DNA
agccacaaagaaaggagttggacc

SEQ ID NO: 47 - DNA, 22 bp, synthetic construct
  misc_feature 1..22: note = hSOX10 forward primer
  source 1..22: mol_type = other DNA
ccacgaggtaatgtccaacatg

SEQ ID NO: 48 - DNA, 18 bp, synthetic construct
  misc_feature 1..18: note = hSOX10 reverse primer
  source 1..18: mol_type = other DNA
cattgggcggcaggtact

(19) United States
(12) Patent Application Publication
(10) Pub. No.: US 2024/0197789 A1
(43) Pub. Date: Jun. 20, 2024

(54) TREATMENT OF CANAVAN DISEASE

(71) Applicant: CITY OF HOPE, Duarte, CA (US)

(72) Inventors: Yanhong SHI, Arcadia, CA (US); Jianfei CHAO, Temple City, CA (US); Wendong LI, Duarte, CA (US)

(73) Assignee: CITY OF HOPE, Duarte, CA (US)

(21) Appl. No.: 18/488,859

(22) Filed: Oct. 17, 2023

Related U.S. Application Data
(63) Continuation of application No. 16/311,995, filed on Dec. 20, 2018, now abandoned.
(60) Provisional application No. 62/353,515, filed on Jun. 22, 2016.

(51) Int. Cl. A61K 35/30; A61K 38/00; C12N 5/074; C12N 5/0797

(52) U.S. Cl. CPC A61K 35/30; C12N 5/0623; C12N 5/0696; A01K 2217/075; A01K 2227/105; A01K 2267/0318; A61K 38/00; C12N 2501/602; C12N 2501/603; C12N 2501/604; C12N 2501/606; C12N 2501/608; C12N 2506/1307; C12N 2506/45; C12N 2510/00; C12N 2740/16043

(57) ABSTRACT

Disclosed herein are methods of treating Canavan disease in a subject through restoring ASPA enzymatic activities in the subject by expressing exogenous wild type ASPA gene in the brain of the subject. Also disclosed are a process of producing neural precursor cells, including NPCs, glial progenitor cells and oligodendroglial progenitor cells, which express an exogenous wild type ASPA gene and the neural precursor cells produced by this process.

### PRIORITY CLAIM

This application is a continuation of U.S. patent application Ser. No. 16/311,995, filed Dec. 20, 2018, which is a U.S. national phase application of International Patent Application No. PCT/US2017/038853, filed Jun. 22, 2017, which claims priority to U.S. Provisional Patent Application No. 62/353,515, filed Jun. 22, 2016, all of which are incorporated herein by reference in their entirety, as if fully set forth herein.

### STATEMENT OF GOVERNMENT FUNDING

This invention was made with government support under grant number TR2-01832 and RB4-06277 awarded by California Institute for Regenerative Medicine.

### SEQUENCE LISTING

This application contains a ST.26 compliant Sequence Listing, which was submitted in XML format via Patent Center, and is hereby incorporated by reference in its entirety. The XML copy, created on Jan. 12, 2024, is named 0544358150US02.xml and is 61,000 bytes in size.

### BACKGROUND

Canavan disease (CD) is a devastating neurological disease with symptoms that appear in early infancy and progress rapidly. Possible symptoms include mental retardation, loss of acquired motor skills, feeding difficulties, abnormal muscle tone, unusually large head, paralysis, blindness, and hearing loss. Canavan disease is caused by genetic mutations in the aspartoacylase (ASPA) gene, which encodes a metabolic enzyme synthesized by oligodendrocytes in the brain15. ASPA breaks down N-acetyl-aspartate (NAA), a highly abundant amino acid derivative in the brain. The cycle of production and breakdown of NAA appears to be critical for maintaining the white matter of the brain, which consists of nerve fibers covered by myelin. Signs of Canavan disease include lack of ASPA activity, accumulation of NAA in the brain, and spongy degeneration and demyelination of the brain.

In recent years, more studies have begun to be devoted to the development of potential therapies for Canavan disease. Gene therapy for Canavan disease using human ASPA-expressing non-viral vectors or AAV vectors has been reported both in Canavan disease animal models and in clinical trials with Canavan disease patients16-22. These studies demonstrate that the ASPA vector is well-tolerated in both animals and human subjects and varied biochemical and clinical improvements have been observed16-22. Modified ASPA protein has been tested for potential use as enzyme replacement therapy in a Canavan disease mouse model23. Elevated ASPA activity and reduced NAA levels were observed in brains of treated mice23. Whether it could improve spongiform degeneration, demeylination and motor function defects remains to be tested. Lithium has been evaluated in Canavan disease animal model and patients and has been shown to reduce NAA level and induce a trend toward normal myelin development in CD-like rats and CD patients. However, it fails to improve the motor function of Canavan patients. Dietary glyceryl triacetate and triheptanoin have been tested in Canavan disease animal models, with improvement in myelination and motor performance observed in treated mice. However, no reduction in NAA levels and only partial amelioration of pathological features were observed. To date, none of these approaches has resulted in complete rescue of the disease phenotypes. There is neither a cure nor a standard treatment for this disease yet.

Accordingly, there is a need in the art to provide an effective therapy to Canavan disease. The invention disclosed herein satisfies this need.

### SUMMARY

In one aspect, this disclosure relates to a method of treating Canavan disease in a subject. The method entails restoring ASPA enzymatic activities in the subject by expressing exogenous wild type ASPA gene in the brain of the subject. In some embodiments, the ASPA enzymatic activities are restored by providing wild type ASPA-expressing neural precursor cells, including neural progenitor cells (NPCs), glial progenitor cells, and oligodendroglial progenitor cells, to the brain of the subject.

In a related aspect, this disclosure relates to neural precursor cells, including NPCs, glial progenitor cells, and oligodendroglial progenitor cells, which express an exogenous wild type ASPA gene produced by a process comprising the steps of reprogramming or converting somatic cells isolated from a subject suffering from Canavan disease into induced pluripotent stem cells (iPSCs), introducing wild type ASPA gene in the reprogrammed or converted iPSCs to obtain genetically corrected iPSCs which express wild type ASPA, and differentiating the genetically corrected iPSCs into neural precursor cells, including NPCs, glial progenitor cells and oligodendroglial progenitor cells. Alternatively, the neural precursor cells, including NPCs, glial progenitor cells and oligodendroglial progenitor cells, which express an exogenous wild type ASPA gene are produced by a process comprising the steps of reprogramming or converting somatic cells isolated from a subject suffering from Canavan disease into induced pluripotent stem cells (iPSCs), differentiating the reprogrammed iPSCs into neural precursor cells, and introducing wild type ASPA gene into the neural precursor cells to obtain genetically corrected neural precursor cells which express wild type ASPA.

In another aspect, this disclosure relates to a method of treating Canavan disease in a subject. The method entails the steps of reprogramming or converting somatic cells isolated from the subject suffering from Canavan disease into induced pluripotent stem cells (iPSCs), introducing wild type ASPA gene in the reprogrammed or converted iPSCs to obtain genetically corrected iPSCs which express wild type ASPA, differentiating the genetically corrected iPSCs into neural precursor cells, including NPCs, glial progenitor cells and oligodendroglial progenitor cells, and transplanting the neural precursor cells into the brain of the subject. Alternatively, the method entails the steps of reprogramming or converting somatic cells isolated from the subject suffering from Canavan disease into induced pluripotent stem cells (iPSCs), differentiating the iPSCs into neural precursor cells, including NPCs, glial progenitor cells and oligodendroglial progenitor cells, introducing wild type ASPA gene into the neural precursor cells to obtain genetically corrected neural precursor cells which express wild type ASPA, and transplanting the genetically corrected neural precursor cells into the brain of the subject.

In some embodiments, the somatic cells include but are not limited to fibroblasts, blood cells, urinary cells, adipocytes, keratinocytes, dental pulp cells and other easily accessible somatic cells. In some embodiments, the somatic cells isolated from the subject suffering from Canavan disease are converted into iPSCs in the presence of one or more reprogramming factors comprising OCT4, SOX2, KLF4, LIN28, p53 shRNA and MYC (such as c-MYC and L-MYC). In some embodiments, the reprogramming is carried out via episomal reprogramming or viral transduction.

In another aspect, this disclosure relates to a method of producing wild type ASPA-expressing neural precursor cells which serve as a source of the ASPA enzyme as well as neural precursors to generate WT ASPA-expressing oligodendrocyte progenitor cells (OPCs) and oligo dendrocytes for treating Canavan disease. The method includes the steps of reprogramming or converting somatic cells isolated from a subject suffering from Canavan disease into induced pluripotent stem cells (iPSCs), introducing wild type ASPA gene in the reprogrammed or converted iPSCs to obtain genetically corrected iPSCs which express wild type ASPA, and differentiating the genetically corrected iPSCs into neural precursor cells, including NPCs, glial progenitor cells, and oligodendroglial progenitor cells. Alternatively, the method includes the steps of reprogramming or converting somatic cells isolated from a subject suffering from Canavan disease into induced pluripotent stem cells (iPSCs), differentiating the iPSCs into neural precursor cells, including NPCs, glial progenitor cells, and oligodendroglial progenitor cells, and introducing wild type ASPA gene in the precursor cells to obtain genetically corrected precursor cells which express wild type ASPA.

### BRIEF DESCRIPTION OF THE DRAWINGS

This application contains at least one drawing executed in color. Copies of this application with color drawing(s) will be provided by the Office upon request and payment of the necessary fees.

FIG. 1 illustrates characterization of iPSCs derived from WT and CD patient fibroblasts. Expression of human ESC markers in CD iPSCs. CD iPSCs derived from three patients, CD patient 1, CD patient 2, and CD patient 3, expressed human pluripotency factors OCT4 and NANOG, and the human ESC cell surface marker SSEA4, TRA-1-60 and TRA-1-81. The WT iPSCs derived from IMR90 cells were included as the WT control. Scale bar: 100 μm.

FIGS. 2A-2D illustrate that CD patient iPSCs and ASPA iPSCs express human ESC markers. FIG. 2A shows RT-PCR analysis of endogenous (endo) OCT4, SOX2, and NANOG expression in WT, CD and ASPA iPSCs. CD patient fibroblasts (fib) were included as negative controls. Actin was included as a loading control. FIGS. 2B and 2C show RT-PCR analysis of exogenous (exo) reprogramming factors in WT, CD and ASPA iPSCs. Human ESCs were included as a negative control and plasmid DNAs expressing individual factors were included as a positive control. FIG. 2D shows the karyotype of control and CD iPSCs.

FIGS. 3A-3F illustrate characterization of mutations in CD iPSCs and validation of the pluripotency of iPSCs. FIG. 3A shows that CD iPSCs contained patient-specific ASPA mutations. FIG. 3B shows validation of iPSC pluripotency in vitro. CD1, CD2 and CD3 iPSCs were able to differentiate into all three germ layers, SOX17-positive endoderm, SMA-positive mesoderm, and TUJ1-positive ectoderm in EB formation assays. WT iPSCs derived from IMR90 cells were included as a control. FIGS. 3C and 3D show validation of iPSC pluripotency in vivo. After injection into immunodeficient NSG mice, CD1 iPSCs (3C), as well as CD2 iPSCs and CD3 iPSCs (3D) were able to form teratomas containing tissues characteristic of each of the three germ layers. Scale bar: 100 μm for FIGS. 3B-3D. FIGS. 3E & 3F show bisulfite sequencing analysis of OCT4 and NANOG promoter regions in parental CD1 fibroblasts (fib) and CD1 iPSCs. Open and closed circles indicate unmethylated and methylated CpGs, respectively.

FIGS. 4A-4M illustrate that the ASPA iPSCs contain the WT ASPA gene and express pluripotency factors. FIGS. 4A, 4D, and 4G show that genomic DNA sequencing confirms the presence of the WT ASPA sequence in ASPA1, ASPA2, and ASPA3 iPSCs. FIGS. 4B, 4E, and 4H show expression of the human pluripotency factors OCT4 and NANOG in ASPA1, ASPA2, and ASPA3 iPSCs. Nuclei Dapi staining is shown in blue. Scale bar: 100 μm. FIGS. 4C, 4F, and 4I show expression of human ESC cell surface markers SSEA4, TRA-1-60 and TRA-1-8 in ASPA iPSCs. Nuclei Dapi staining is shown in blue. FIG. 4J shows that genomic DNA sequencing confirms the presence of WT ASPA sequence in ASPA1, ASPA2 iPSCs. FIGS. 4K and 4L show expression of transduced ASPA in the ASPA1 iPSCs as revealed by RT-PCR (4K) and Western blot analyses (4L). GAPDH and Tubulin were included as loading controls. FIG. 4M shows validation of the developmental potential of the ASPA1 iPSCs, ASPA2 iPSCs and ASPA3 iPSCs in vivo. After injection into immunodeficient NSG mice, the ASPA1 iPSCs, ASPA2 iPSCs and ASPA3 iPSCs were able to form teratomas containing tissues characteristic of each of the three germ layers. Scale bar: 100 μm.

FIGS. 5A-5G illustrate characterization of the ASPA1 NPCs. FIG. 5A shows immunostaining for NPC markers PAX6, SOX2, NCAD, SOX1, and NESTIN in NPCs derived from the WT, CD1, and ASPA1 iPSCs. Nuclei Dapi staining is shown in blue. Scale bar: 50 μm. FIG. 5B shows expression of ASPA and NPC markers in the ASPA1 NPCs as revealed by RT-PCR. The WT and CD1 NPCs were included as controls. GAPDH was included as a loading control. FIG. 5C shows lack of expression of pluripotency factors in the WT, CD1, and ASPA1 NPCs as revealed by RT-PCR. The WT iPSCs and CD1 fibroblasts were included as positive and negative controls, respectively. FIG. 5D shows that the ASPA NPCs displayed potent ASPA enzymatic activity, compared to the CD1 NPCs. Error bars are s.d. of the mean (n=5 repeats). * p<0.05 by Student's t-test. FIGS. 5E and 5F show immunostaining pre-OPCs derived from CD1 NPCs or ASPA1 NPCs using OLIG2 and NKX2.2 (5E) or live staining of OPCs derived from CD1 NPCs or ASPA1 NPCs using 04 (5F). Scale bar: 100 μm for 5E and 50 μm for 5F. FIG. 5G shows FACS analysis of CD1 NPCs and ASPA1 NPCs.

FIGS. 6A and 6B illustrate that the ASPA1 NPCs survived and expressed OLIG2 in transplanted CD mouse brains. FIG. 6A shows that the ASPA1 NPCs were transplanted into neonatal CD mice. One month after transplantation, the mouse brains were harvested and immunostained with antibodies for human nuclear antigen and OLIG2. Scale bar: 100 μm. FIG. 6B shows that transplantation of the ASPA-CD1 NPCs rescued the motor function deficits in CD mice, as revealed in rotarod test one month after transplantation. Error bars are s.e. of the mean (n=6 mice). *** p<0.001 by Student's t-test.

FIGS. 7A-7C illustrate that the ASPA1 NPCs gave rise to OLIG2+ cells in transplanted CD mouse brains. FIG. 7A shows that the ASPA1 NPCs were transplanted into neonatal CD mice. Three months after transplantation, the mouse brains were harvested and immunostained with antibodies for human nuclear antigen (green) and OLIG2 (red). FIG. 7B shows that three months after transplantation, the mouse brains were harvested and immunostained with antibodies for human nuclear antigen (green) and MBP (red). The enlarged images of the human nuclear antigen (green) and MBP (red)-double positive cells pointed by the arrows were shown in the lower panels. FIG. 7C shows that the engrafted human cells differentiated into GFAP+(red) glial cells. Scale bar: 100 μm for panel A, 63 μm for the upper panels and 10 μm for the lower panels of panel B, and 63 μm for panel C.

FIGS. 8A-8E illustrate that the ASPA1 NPCs reduced NAA level and vacuolation in CD mice. FIG. 8A shows elevated ASPA enzymatic activity in CD mice three months after ASPA1 NPC transplantation. Error bars are s.d. of the mean (n=5 mice). FIGS. 8B and 8C show reduced NAA level in ASPA1 NPC-transplanted CD mouse brains measured using NMR. Error bars are s.e. of the mean (n=5 mice). FIGS. 8D and 8E show reduced vacuolation in ASPA1 NPC-transplanted CD mouse brains. H&E staining of thalamus, cerebellum and brain stem in control CD mice and ASPA1 NPC-transplanted CD mice is shown in panel 8D and quantification of percent vacuolation is shown in panel 8E. Scale bar: 100 μm. Error bars are s.e. of the mean (n=6 mice). ** p<0.01, and *** p<0.001 by Student's t-test for all quantification.

FIGS. 9A-9E illustrate that the ASPA1 NPCs improved myelination and motor function in CD mice. FIGS. 9A and 9B show rescued myelination in ASPA1 NPC-transplanted CD mice. Intact and thick myelin sheaths were detected in brains of 3-month-old wild type (WT) mice, whereas splitting and thinner myelin sheaths were seen in brains of littermate CD mice. Myelin sheaths in CD mice transplanted with the ASPA1 NPCs for three months appeared more intact and thicker. Images of brain stem region are shown. Scale bar: 1 μm. Arrows point to myelin sheaths.

FIG. 9C shows transplantation of the ASPA1 NPCs rescued the weight loss in CD mice. FIGS. 9D and 9E show that transplantation of the ASPA1 NPCs rescued the motor function deficits in CD mice, as revealed in rotarod (9D) or hanging wire (9E) test. Error bars are s.e. of the mean (n=6 mice). * p<0.05, ** p<0.01, and *** p<0.001 by Student's t-test for 9B-9E.

FIGS. 10A-10D illustrate that the ASPA2 NPCs and ASPA3 NPCs exhibited potent ASPA enzymatic activity in vitro. FIG. 10A shows expression of the NPC markers NESTIN and SOX1 in the ASPA2 NPCs and ASPA3 NPCs by immunostaining. Nuclei Dapi staining is shown in blue in the merged images. Scale bar: 100 μm. FIG. 10B shows lack of expression of the pluripotency factors OCT4 and NANOG in the ASPA2 NPCs and ASPA3 NPCs as revealed by RT-PCR. ESCs and CD2, CD3 fibroblasts (Fib) were included as positive and negative controls, respectively. FIG. 10C shows FACS analysis of the ASPA2 NPCs and ASPA3 NPCs. FIG. 10D shows that the ASPA2 NPCs and ASPA3 NPCs displayed significantly increased ASPA enzymatic activity, compared to CD2 NPCs and CD3 NPCs. Error bars are s.e. of the mean (n=6 repeats). *** p<0.001 by Student's t-test.

FIGS. 11A-11C demonstrate that the Aspanur7/nur7/Rag2−/− (Aspanur7/Rag2-+) mice exhibited similar pathological features to the parental Aspanur7/nur7 (Aspanur7) mice. FIG. 11A shows similar ASPA enzymatic activity in the brains of Aspanur7/Rag2−/− mice and the parental Aspanur7 mice. Error bars are s.d. of the mean (n=5 mice). *** p<0.001 by Student's t-test. FIGS. 11B and 11C show similar vacuolation in the brains of Aspanur7/Rag2^{-/-} mice and the parental Aspanur7 mice. H&E staining of thalamus, cerebellum and brain stem in control CD mice and ASPA-CD1 NPC-transplanted CD mice is shown in 11B and quantification is shown in 11C. Scale bar: 100 μm. Error bars are s.e. of the mean (n=6 mice).

FIGS. 12A-12B demonstrate that the ASPA1 NPCs gave rise to OLIG2+oligodendroglial lineage cells and MBP+oligodendrocytes in CD mouse brains. FIG. 12A shows quantification of the percentage of human nuclear antigen (hNu)+ and OLIG2+ cells from total grafted human cells in the ASPA1 NPC-transplanted CD brains three months after engraftment. The ASPA1 NPCs were transplanted into neonatal CD mice. Three months after transplantation, the mouse brains were harvested and immunostained with antibodies for human nuclear antigen (green) and MBP (red). The percentage of hNu+ and OLIG2+ cells out of total hNu+ cells were shown. Error bars are s.e. of the mean (n=6 mice). FIG. 12B is an orthogonal view to show co-staining for human nuclear antigen and MBP in the ASPA1 NPC-transplanted CD mouse brains. Scale bar: 10 μm.

FIG. 13 shows that the ASPA1 NPCs can differentiate into GFAP+ cells in transplanted CD mouse brains. The ASPA1 NPCs were transplanted into neonatal CD mice. Three months after transplantation, the mouse brains were harvested and immunostained with antibodies for human nuclear antigen (green) and GFAP (red). Scale bar: 63 μm.

FIGS. 14A and 14B show that the ASPA2 and ASPA3 NPCs survived and expressed OLIG2 and GFAP in transplanted CD mouse brains. The ASPA2 NPCs and ASPA3 NPCs were transplanted into neonatal CD mice. FIG. 14A shows that three months after transplantation, the mouse brains were harvested and immunostained with antibodies for human nuclear antigen (green) and OLIG2 (red). Scale bar: 25 μm. FIG. 14B shows that three months after transplantation, the mouse brains were harvested and immunostained with antibodies for human nuclear antigen (green) and GFAP (red). Scale bar: 50 μm.

FIGS. 15A-15G show that the ASPA2 and ASPA3 NPCs reduced vacuolation and improved motor function in CD mice. FIG. 15A shows that CD mice transplanted with the ASPA2 NPCs or ASPA3 NPCs were immunostained for human nuclear antigen (green) and MBP (red). The enlarged images of the human nuclear antigen (green) and MBP (red)-double positive cells pointed by the arrows were shown in the lower panels. Scale bar: 50 μm for the upper panels and 10 μm for the lower panels. FIG. 15B is an orthogonal view to show co-staining for human nuclear antigen and MBP in the ASPA2 NPC or ASPA3 NPC-transplanted CD mouse brains. Scale bar: 10 μm. FIG. 15C demonstrates elevated ASPA activity in the thalamus, cerebellum and brain stem of CD mice three months after transplantation with the ASPA2 NPCs or ASPA3 NPCs. Error bars are s.e. of the mean (n=6 mice). * p<0.05 by Student's t-test. FIGS. 15D and 15E show reduced vacuolation in the ASPA2 NPCs or ASPA3 NPC-transplanted CD mouse brains. Quantification of percent vacuolation is shown in 15D. Error bars are s.e. of the mean (n=6 mice). ** p<0.01 and *** p<0.001 by Student's t-test. H&E staining of the thalamus, cerebellum and brain stem in control CD mice or CD mice transplanted with the ASPA2 NPCs or ASPA3 NPCs is shown in 15E. Scale bar: 100 μm.

FIGS. 15F and 15G show that the ASPA2 NPCs or ASPA3 NPCs rescued the motor function deficits in the transplanted CD mice in rotarod (15F) or hanging wire (15G) test. Error bars are s.e. of the mean (n=6 mice). ** p<0.01 and *** p<0.001 by Student's t-test.

FIGS. 16A-16B demonstrate that no tumor formation in the brains of the ASPA1 NPC-transplanted CD mice. Tumor was analyzed through H&E staining 10 months after transplantation with the ASPA1 NPCs. No typical tumor tissue was found in the ASPA1 NPC-transplanted CD mouse brains. Scale bar: 100 μm.

FIGS. 17A-17B show low Ki67 index of human engrafted cells at 3 months after transplantation. FIG. 17A shows that the ASPA-CD1 NPCs were transplanted into CD mouse brains. Three months after transplant, the engrafted brains were immunostained for human nuclear antigen (green) and Ki67 (red). Scale bar: 50 μm. FIG. 17B shows quantification of the percent of human nuclear antigen (hNu)+ and Ki67+ cells out of total hNu+ cells in the ASPA1 NPC-transplanted CD mouse brains. Error bars are s.e. of the mean (n=6 mice).

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

Stem cell technology holds great promise for the treatment of neurological disorders. However, the availability of expandable sources of stem cells is a critical issue in moving stem cell technology to bedside. Human iPSCs derived by reprogramming adult human fibroblasts could provide a continuous and autologous donor source for the generation of specific somatic cell types and tissues from individual patients1-4. Patient-specific iPSCs could provide an unlimited reservoir of disease cell types that otherwise would not be available. Furthermore, patient-specific iPSCs are tailored to specific individuals, therefore could reduce the potential for immune rejection. Moreover, recent work demonstrates the feasibility to generate genetically corrected iPSCs from both mice and humans by viral transduction of the wild type (WT) gene or site-specific gene editing. These iPSCs could provide exciting prospects for cell therapy and for studying disease mechanisms.

The combination of gene therapy with cell therapy provides tremendous hope for a variety of genetic disorders. The therapeutic combination of patient-specific iPSCs with gene therapy provides an opportunity to correct gene defects in vitro, and these genetically-repaired iPSCs can then be appropriately characterized to ensure that the genetic correction is precise, thereby reducing safety concerns associated with direct gene therapy, such as random gene insertions5,6.

Considerable interest has been aroused in generating iPSCs from patients of neurodegenerative diseases since the breakthrough development of the iPSC technology. These patient-specific iPSCs offer many opportunities for disease modeling, drug discovery, and cell replacement therapy. On the other hand, extensive efforts have been made to develop and optimize methods to differentiate pluripotent stem cells into different neural lineages. These methods allow the generation of neural cell types from genetically corrected iPSCs for cell replacement therapy.

Demyelinating diseases stand out as a particularly promising target for cell-based therapy of central nervous system disorders because remyelination can be achieved with a single cell type, and transplanted myelinogenic cells do not need to integrate into complex neuronal networks7. Indeed, the myelinogenic potential of rodent and human pluripotent stem cell derivatives have been well documented in various animal models8-14. The widespread myelination that can be observed in animal models supports the idea that cell therapy provides a potential therapeutic approach in dysmyelinating and demeylinating diseases.

As disclosed herein, iPSC-based cell therapy approach is combined with gene therapy approach to generate genetically-corrected patient iPSCs that express the wild type ASPA gene (ASPA iPSCs). Subsequently, the ASPA iPSCs are differentiated into neural precursor cells, including NPCs, glial progenitor cells, oligodendroglial progenitor cells, and the therapeutic potential thereof is assessed in an immune-deficient Canavan disease mouse model.

Thus, disclosed herein is a method of treating Canavan disease in a subject. The method combines patient-specific iPSCs with gene therapy to develop genetically-corrected patient iPSCs that express the WT ASPA gene. The ASPA iPSCs were differentiated into NPCs. Alternatively, genetic correction can occur at the neural precursor cells level, that is, reprogrammed iPSCs derived from a patient are differentiated into neural precursor cells, and then the wild type ASPA gene is introduced into the neural precursor cells to generate genetically-corrected neural precursor cells. The ability of these neural precursors to alleviate the disease phenotypes of CD was tested in a CD mouse model, as demonstrated in the working examples. Also, the preclinical efficacy for neural precursor cells derived from genetically corrected patient iPSCs to serve as a therapeutic candidate for CD is demonstrated in the working examples.

Because CD is caused by genetic mutation in the ASPA gene, the CD patient iPSCs or neural precursor cells are genetically corrected by introducing the WT ASPA gene through lentiviral transduction. The resultant ASPA iPSCs are differentiated into neural precursor cells. The ASPA neural precursor cells exhibit potent ASPA activity, in contrast to CD iPSC-derived CD NPCs that exhibit almost no detectable ASPA activity. The ASPA neural precursor cells are transplanted into a CD mouse model that exhibits key pathological phenotypes of CD, including loss of ASPA activity, elevated NAA levels, and extensive sponge degeneration in various brain regions. The transplanted ASPA neural precursor cells are able to survive after transplantation and differentiate into oligodendrogial lineage cells. Moreover, the transplanted cells are able to exhibit potent ASPA enzymatic activity and reduce NAA levels and sponge degeneration in the brains of transplanted CD mice. Transplantation of the ASPA-CD neural precursor cells also can rescue weight loss and behavioral defects of CD mice. Importantly, no tumorigenesis or other adverse effect is observed in the transplanted mice. These results indicate that the ASPA-CD neural precursor cells could serve as potential cell replacement therapeutic candidate for CD.

There is no cure for CD and treatment for CD is symptomatic only. The application of cell therapy is gaining great momentum because it could have broad therapeutic impact. The grafted cells could not only serve as a sustained source of the missing enzymes but also offer a replacement of the lost cells in the host. As disclosed herein, iPSC-derived neural precursor cells can be a cell therapy candidate for CD because neural precursor cells have the capacity to differentiate into oligodendroglial lineage cells that are lost in CD. Specifically, neural precursor cells derived from the genetically corrected, WT ASPA-expressing CD iPSCs can be used because the ASPA-CD neural precursor cells can not only replace the lost oligodendroglial lineage cells but also reconstitute the missing ASPA enzyme. A major obstacle for moving cell therapy to humans is to have enough cells for transplantation into patients. iPSCs can provide an unlimited source of cells that are otherwise not possible to obtain for cell replacement therapy due to their easy accessibility and extensive expandability. Moreover, patient-specific iPSCs can provide a source of autologous cells that may avoid immunogenicity associated with allogeneic cell transplantation, therefore offering an option for the treatment of human diseases using cell replacement therapy.

The differentiated product of iPSCs has not been shown to form teratomas. To address the safety concern associated with potential development of teratoma, it is important to make sure the final iPSC-derived product does not include the undifferentiated cells. The method disclosed herein differentiates human iPSCs into neural precursor cells in very high efficiency. As demonstrated in the working examples, FACS analysis of the ASPA1 NPCs showed that more than 98% cells are positive for CD133, a cell surface marker for human NPCs. In contrast, only 0.054% cells are positive for SSEA4, a human ESC surface marker. For ASPA2 NPCs and ASPA3 NPCs, high percentage of CD133-positive cells was also detected. Moreover, cells that were sorted through both positive selection for CD133 and negative election for SSEA4 for ASPA2 and ASPA3 NPC transplantation were used to ensure no contamination of pluripotent stem cells.

Gene therapy is a promising clinical option for CD. Both pre-clinical and clinical gene therapy studies have been performed by delivering the wild type human ASPA gene into CD animal models or CD patients and encouraging progress has been made16-22. However, only partial amelioration of the pathological features has been achieved, presumably because the dying oligodendrocytes cannot be replaced by gene therapy. Targeted delivery of the WT ASPA into the precursors of oligodendrocytes led to improved outcome, presumably because the WT ASPA was reconstituted in oligodendroglial lineage cells, supporting the importance of targeting oligodendrocytes in therapeutic design for CD. Nevertheless, gene therapy alone does not have the capacity to replace the lost host cells and could not provide potential trophic supports that may help to impede disease progression and facilitate recuperation. Besides gene therapy, recent studies demonstrated that knocking out the NAA synthase Nat8L (N-acetyltransferase-8 like) gene could prevent the ASPAnur7/nur7 mice from developing certain pathological aspects of CD by abolishing their ability to generate NAA.

Because CD has a deficiency in both brain ASPA enzyme and oligodendrocytes, an ideal strategy for the treatment of CD would be to restore brain ASPA activity and replace dying oligodendrocytes using combined cell therapy and gene therapy. The restored ASPA activity could in turn reduce NAA level. Transplantation of NPCs expressing the WT human ASPA gene and possessing the ability to differentiate into OPCs and oligodendrocytes offers an appealing therapeutic approach for CD by reconstituting both the missing ASPA enzyme and the lost oligodendroglial lineage cells. Indeed, mouse neural precursor cells transduced with the WT ASPA gene were able to survive and differentiate into oligodendrocytes, and exhibit detectable ASPA activity after transplanting into the brains of CD mice41, suggesting that neural precursor cells can be used as a potential source of cell therapy for CD. However, this previous study used mouse cells, which are not clinically applicable41. Moreover, increased ASPA activity was only detected at 3 weeks after transplantation, the ASPA activity became undetectable at 5 weeks after transplantation, presumably because of the short term in vivo gene expression from a retroviral vector41. Furthermore, the effect of the transplanted mouse NPCs on the pathological phenotypes of CD was not studied in the previous study41. As detailed in the embodiments, CD patient iPSCs are combined with gene therapy approach to generate genetically-corrected human ASPA iPSCs by transducing CD iPSCs with lentivirus expressing the WT ASPA gene. These iPSCs are subsequently differentiated into ASPA neural precursor cells. Alternatively, the CD patient iPSCs are differentiated into neural precursor cells and then the WT ASPA gene is introduced into neural precursor cells. The resultant ASPA neural precursor cells served as a source of the ASPA enzyme and as neural precursors to generate WT ASPA-expressing oligodendrocyte progenitor cells (OPCs) and oligodendrocytes. Even three months after transplantation, robust ASPA enzymatic activity in the transplanted CD mouse brains was detected. More importantly, substantial rescue of major pathological phenotypes and behavioral defects in CD mice, including substantially elevated ASPA enzymatic activity, reduced NAA level and vacuolation, enhanced myelination, increase body weight and improved motor function, were detected. These patient-specific and genetically-corrected ASPA neural precursor cells therefore serve as an ideal cell replacement therapy for CD patients.

Existing therapy for Canavan disease resulted in improved functional recovery to certain extent; however, none have resulted in complete correction of the pathological features associated with the disease. The method disclosed herein, by combining cell therapy with gene therapy, providing enzyme replacement and cell replacement, provides a novel therapy for Canavan disease that exhibits much improved clinical outcome.

The use of an immunologically deficient murine model of CD as disclosed herein permitted the transplant of human neural precursor cells without immune rejection. There are only minimum amino acid differences between the WT and the CD variants of ASPA. In the clinical trial of gene therapy for CD using an adeno-associated viral vector expressing the WT ASPA gene, no long-term adverse events were observed in CD patients. Therefore, an additional advantage is that no immune rejection results from the expression of the WT ASPA gene in the ASPA-CD neural precursor cells.

In one aspect, this disclosure relates to a method of treating Canavan disease in a subject. The method entails restoring ASPA enzymatic activities in the subject by expressing exogenous wild type ASPA gene in the brain of the subject. In some embodiments, the ASPA enzymatic activities are restored by transplanting ASPA neural precursor cells in the brain of the subject. These ASPA neural precursor cells serve as a source of the ASPA enzyme as well as neural precursors to generate WT ASPA-expressing oligodendrocyte progenitor cells (OPCs) and oligo dendrocytes. As detailed in this disclosure, ASPA neural precursor cells can be derived from patient-specific iPSCs. For example, the method further includes the steps of reprogramming or converting somatic cells isolated from the subject suffering from Canavan disease into induced pluripotent stem cells (iPSCs), introducing wild type ASPA gene in the reprogrammed or converted iPSCs to obtain genetically corrected iPSCs which express wild type ASPA, and differentiating the genetically corrected iPSCs into neural precursor cells, including NPCs, glial progenitor cells and oligodendroglial progenitor cells. Alternatively, the method further includes the steps of reprogramming or converting somatic cells isolated from the subject suffering from Canavan disease into induced pluripotent stem cells (iPSCs), differentiating the iPSCs into neural precursor cells, including NPCs, glial progenitor cells and oligodendroglial progenitor cells, and introducing wild type ASPA gene in the neural precursor cells to obtain genetically corrected neural precursor cells which express wild type ASPA.

In some embodiments, the somatic cells include but are not limited to fibroblasts, blood cells, urinary cells, adipocytes, keratinocytes, dental pulp cells, and other easily accessible somatic cells. In some embodiments, the somatic cells isolated from the subject suffering from Canavan disease are converted into iPSCs in the presence of one or more reprogramming factors comprising OCT4, SOX2, KLF4, LIN28, p53 shRNA and MYC (such as c-MYC and L-MYC). In some embodiments, the reprogramming is carried out via episomal reprogramming or viral transduction. It is within the purview of one skilled in the art to select a reprogramming technique to convert the patient somatic cells into iPSCs. In some embodiments, the wild type ASPA gene is introduced into the reprogrammed iPSCs by transducing the reprogrammed iPSCs with a vector comprising the exogenous wild type ASPA gene. It is within the purview of one of ordinary skill in the art to select a suitable vector and promoter to express the wild type ASPA gene after transduction. In some embodiments, the wild type ASPA gene is introduced by gene editing technology (such as the CRISPR/Cas9 technology).

In another aspect, this disclosure relates to a method of treating Canavan disease in a subject. The method entails the steps of reprogramming or converting somatic cells isolated from the subject suffering from Canavan disease into induced pluripotent stem cells (iPSCs), introducing wild type ASPA gene in the reprogrammed or converted iPSCs to obtain genetically corrected iPSCs which express wild type ASPA, differentiating the genetically corrected iPSCs into neural precursor cells, and transplanting the neural precursor cells into the brain of the subject. In some embodiments, the method entails the steps of reprogramming or converting somatic cells isolated from the subject suffering from Canavan disease into induced pluripotent stem cells (iPSCs), differentiating the iPSCs into neural precursor cells, introducing wild type ASPA gene in the neural precursor cells to obtain genetically corrected neural precursor cells which express wild type ASPA, and transplanting the genetically corrected neural precursor cells into the brain of the subject.

In some embodiments, the somatic cells include but are not limited to fibroblasts, blood cells, urinary cells, adipocytes, keratinocytes, dental pulp cells, and other easily accessible somatic cells. In some embodiments, the somatic cells isolated from the subject suffering from Canavan disease are converted into iPSCs in the presence of one or more reprogramming factors comprising OCT4, SOX2, KLF4, LIN28, p53 shRNA and MYC (such as c-MYC and L-MYC).

In some embodiments, the reprogramming is carried out via episomal reprogramming or viral transduction. It is within the purview of one skilled in the art to select a reprogramming technique to convert the patient somatic cells into iPSCs. The iPSCs converted from patient somatic cells contain one or more mutations in the ASPA protein. For example, some patients suffering from Canavan disease carry one or more mutations in the ASPA protein, such as A305E, E285A, or G176E mutation, resulting from a codon change of 914C>A, 854A>C, and 527G>A, respectively. Some Canavan disease patients may carry other mutations in different regions of the ASPA protein. Upon introducing wild type ASPA gene into the patient iPSCs, these iPSCs are genetically corrected to express exogenous wild type ASPA protein and exhibit ASPA enzymatic activities.

In some embodiments, the wild type ASPA gene is introduced into the reprogrammed iPSCs by transducing the reprogrammed iPSCs with a vector comprising the exogenous wild type ASPA gene. It is within the purview of one of ordinary skill in the art to select a suitable vector and promoter to express the wild type ASPA gene after transduction. For example, the exogenous wild type ASPA gene can be introduced by transducing the patient iPSCs with a lentivirus comprising the wild type ASPA gene. The ASPA gene mutation in Canavan disease patient iPSCs can also be corrected by gene editing technologies, such as the CRISPR/Cas9 technology. The genetically corrected iPSCs are differentiated in vitro into neural precursor cells, which also express wild type ASPA. After transplanting these ASPA NPCs into the brain of the subject suffering from Canavan disease, the ASPA neural precursor cells can differentiate in vivo into oligodendroglial lineage cells, thereby treating the disease by restoring normal ASPA enzymatic activities. In some embodiments, the genetic correction occurs at the neural precursor cells level in a similar fashion. The CD patient iPSCs are differentiated into neural precursor cells, and then the wild type ASPA gene is introduced to the neural precursor cells by transduction or gene editing, which techniques are known in the art.

In another aspect, this disclosure relates to a method of producing ASPA neural precursor cells which serve as a source of the ASPA enzyme as well as neural precursors to generate WT ASPA-expressing oligodendrocyte progenitor cells (OPCs) and oligo dendrocytes for treating Canavan disease. The ASPA neural precursor cells are derived from patient-specific iPSCs. The method includes the steps of reprogramming or converting somatic cells isolated from a subject suffering from Canavan disease into induced pluripotent stem cells (iPSCs), introducing wild type ASPA gene in the reprogrammed or converted iPSCs to obtain genetically corrected iPSCs which express wild type ASPA, and differentiating the genetically corrected iPSCs into neural precursor cells. Alternatively, the method includes the steps of reprogramming or converting somatic cells isolated from a subject suffering from Canavan disease into induced pluripotent stem cells (iPSCs), differentiating the iPSCs into neural precursor cells, and introducing wild type ASPA gene in the neural precursor cells to obtain genetically corrected neural precursor cells which express wild type ASPA.

In a related aspect, this disclosure relates to neural precursor cells which express an exogenous wild type ASPA gene produced by a process comprising the steps of reprogramming or converting somatic cells isolated from a subject suffering from Canavan disease into induced pluripotent stem cells (iPSCs), introducing wild type ASPA gene in the reprogrammed or converted iPSCs to obtain genetically corrected iPSCs which express wild type ASPA, and differentiating the genetically corrected iPSCs into neural precursor cells. Alternatively, the process comprises the steps of reprogramming or converting somatic cells isolated from a subject suffering from Canavan disease into induced pluripotent stem cells (iPSCs), differentiating the iPSCs into neural precursor cells, and introducing wild type ASPA gene in the neural precursor cells to obtain genetically corrected neural precursor cells which express wild type ASPA. As used herein, neural precursor cells include NPCs, glial progenitor cells and oligodendroglial progenitor cells.

In some embodiments, the somatic cells include but are not limited to fibroblasts, blood cells, urinary cells, adipocytes, keratinocytes, dental pulp cells, and other easily accessible somatic cells. In some embodiments, the somatic cells isolated from the subject suffering from Canavan disease are converted into iPSCs in the presence of one or more reprogramming factors comprising OCT4, SOX2, KLF4, LIN28, p53 shRNA and MYC (such as c-MYC and L-MYC). In some embodiments, the reprogramming is carried out via episomal reprogramming or viral transduction. It is within the purview of one skilled in the art to select a reprogramming technique to convert the patient somatic cells into iPSCs. In some embodiments, the wild type ASPA gene is introduced into the reprogrammed iPSCs by transducing the reprogrammed iPSCs with a vector comprising the exogenous wild type ASPA gene or by genetic editing technology. It is within the purview of one of ordinary skill in the art to select a suitable vector and promoter to express the wild type ASPA gene after transduction.

The terms “treat,” “treating,” and “treatment” as used herein with regards to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. In some embodiments, treating a condition means that the condition is cured without recurrence.

The terms “subject” and “patient” are used interchangeably in this disclosure. In some embodiments, the subject or patient suffers from Canavan disease. In some embodiments, the subject or patient is a mammal. In some embodiments, the subject or patient is a human.

The working examples below further illustrate various embodiments of this disclosure. By no means the working examples limit the scope of this invention.

### Example 1 Materials and Methods

iPSCs generation. For episomal iPSC derivation, IMR90 fibroblasts (Coriell, 190-10) and CD patient fibroblasts (Coriell, GM04268) were reprogrammed using episomal vectors expressing Oct4, Sox2, Klf4, L-Myc, Lin28 and p53 shRNA as described32. Briefly, 5×105 fibroblasts were electroporated with 1.25 μg of each episomal vector, and this day was referred to as day 0. The transfected cells were cultured in fibroblast medium (MEM with NEAA, 15% non-heat-inactivated fetal bovine serum) and medium was changed every other day. Cells were dissociated on day 5 and switched to Essential 8 (E8) medium (Gibco, A15169-01) on day 6. iPSC clones were picked around day 20 and expanded in E8 medium. For viral iPSC derivation, IMR90 fibroblasts or CD patient fibroblasts (Coriell, GM00059, GM00060 and GM04268) were seeded onto 6-well plates at 1×105 cells per well in fibroblast medium. The next day, iPSCs were transduced with freshly prepared viruses of Oct4, Sox2, Klf4 and cMyc as described1, and this day was referred to as day 0. A second round of viral transduction was performed on day 1. On day 5, cells were dissociated and split at 1 to 5. On day 6, cells were switched to E8 medium and medium was changed daily thereafter. iPSC clones were picked around day 20 and expanded in E8 medium.

Embryoid body (EB) formation. To form EBs, iPSCs were dissociated into small clusters using 0.05 mM EDTA and transferred to E8 medium in T25 flasks. After culturing for two days in E8 medium, EB spheres were switched to human ESC medium containing DMEM/F12, 20% knockout serum, 1 mM L-glutamine, but without bFGF. After two weeks, EBs were plated onto gelatin-coated 12-well plates and cultured for another 2 weeks before immunostaining analysis.

Teratoma formation. iPSCs were dissociated with accutase at 1 to 2 dilution in PBS and re-suspended in ice cold mixture of E8 medium and Matrigel (1:1) at the density of 1×107 cells/ml. 100 μl of cell suspension (1×106 cells) were injected subcutaneously into the dorsal flank of immunodeficient Nod Scid Gamma (NSG) mice. Eight to twelve weeks after injection, teratoma was dissected and fixed in formalin. Fixed tissues were embedded in paraffin, sectioned and stained with hemotoxylin and eosin (H&E).

ASPA viral preparation and transduction. To make ASPA-expressing virus, the human ASPA coding sequence was PCR-amplified using human ASPA cDNA (ATCC, MGC-34517) as the template and the PCR product was cloned into the lentiviral vector pSIN-EF2- pur, which was generated by removing Sox2 from pSIN-EF2-Sox2-pur (Addgene, #16577). To package the ASPA-expressing virus, 15 ug of pSIN-EF2-hASPA-pur, 5 ug of VSV-G, 5 ug of REV and 15 ug of MDL were transfected into HEK 293T cells using calcium phosphate transfection method. Forty-eight to seventy-two hr after transfection, virus-containing medium was harvested and filtered through 0.45 μm filter. For viral transduction, freshly harvested ASPA-expressing virus was added to CD iPSCs. Two days after viral transduction, puromycin selection was started and continued for 7 days. The selected ASPA-iPSC clones were expanded and characterized.

Exon sequencing of the ASPA genomic DNA. Genomic DNAs were extracted from CD iPSCs. The primers used for sequencing each exon are list in Table 1 below:

TABLE 1

| Exons | Forward Primer | Reverse Primer |
|---|---|---|
| Exon 1 | 5′-CTC CAC TCA AGG GAA TTC | 5′-ACT GCA TGT ACG GAC ATG |
| | TGT-3′ | AA-3′ |
| Exon 2 | 5′-AGA TTT GGC GAC TGG TTC | 5′-TGC ACC TTC CCT CAT AAC TG- |
| | T-3′ | 3′ |
| Exon 3 | 5′-ACT CTG TTG AAG CAA AGA | 5′-CAG AGC AAG ACT CTG TCT |
| | GA-3′ | CA-3′ |
| Exon 4 | 5′-TTC CAT GAT GCT ACA TGG | 5′-GCA AAT CTG ACC CAG GTT |
| | TT-3′ | CCA-3′ |
| Exon 5 | 5′-TGT TCT CGA ACT CCT GAC | 5′-GCG AAG TGC TGT ATG AGC TA- |
| | CT-3′ | 3′ |
| Exon 6 | 5′-GAT CAA GAC TGG AAA CCA | 5′-GAA GTG TAG TAA GGC AAA |
| | C-3′ | GC-3′ |

Differentiation of human iPSCs into neural progenitor cells (NPCs). NPCs were derived from human iPSCs following an established protocol33. To start neural induction, human iPSCs were dissociated with 0.5 mM EDTA and passaged onto Matrigel-coated plates at 20% confluency in E8 medium. After 24 hr, cells were switched to Neural Induction Medium 1 (NIM-1) containing 50% Advanced DMEM/F12 (Life Technologies, 11330-032), 50% Neurobasal (Life Technologies, 21103-049), N2 (Life Technologies, 17502-048), B27 (Life Technologies, 12587-010), 2 mM GlutaMAX (Life Technologies, 35050-061), 4 uM CHIR99021 (D&C Chemicals, DC9703), 3 uM SB431542 (R&D, 1614) and 2 uM Dorsomorphin (Sigma, P5499). Cells were treated with NIM-1 for 2 days, then switched to Neural Induction Medium 2 (NIM-2) containing 50% Advanced DMEM/F12, 50% Neurobasal, 1×N2, 1×B27, 2 mM GlutaMAX, 4 uM CHIR99021, 3 UM SB431542 and LDN-193189 (Stemgent, 04-0074) for another 5 days. Cells were then dissociated with Accutase (Sigma, A6964), and maintained in Neural Stem cell Maintenance Medium (NSMM) containing 50% DMEM/F12, 50% Neurobasal, 1×N2, 1×B27, 2 mM GlutaMAX, 3 UM CHIR99021, 2 uM SB431542, 20 ng/ml EGF and 20 ng/ml FGF. For the initial 6 passages, NPCs were treated with 10 UM ROCK inhibitor when dissociated. NPCs were transplanted into neonatal mice within 14 passages.

Differentiation of iPSC-derived NPCs into oligodendrocytes. To differentiate iPSC-derived NPCs into oligodendroctyes in vitro, NPCs were switched from NSMM medium (see above) to Neural Induction Medium 3 (NIM-3) containing DMEM/F12, 1×N2, 1×NEAA, 2 mM GlutaMAX, 25 ug/mL Insulin, 0.1 uM RA and 1 UM SAG and cultured in NIM-3 medium for 4 days with daily medium change. Cells were then dissociated and resuspended in NIM-3, and cultured in NIM-3 for 8 days. Thereafter, cells were switched to PDGF medium containing DMEM/F12, 1×N2, 1×NEAA, 2 mM GlutaMAX, 25 ug/mL Insulin (Sigma, 19278), 5 ng/ml HGF (R&D Systems, 294-HG-025), 10 ng/ml PDGF-AA (R&D Systems, 221-AA-050), 10 ng/ml IGF-1 (R&D Systems, 291-G1-200), 10 ng/mL NT3 (Millipore, GF031), 60 ng/mL T3 (Sigma, T2877), 100 ng/ml Biotin (Sigma, 4639), and 1 μM cAMP (Sigma, D0260), for the next 10 days, with medium change every two days. Cells were then attached onto matrigel-coated 6-well plates, and cultured in glial medium containing DMEM/F12, 1×N2, 1×NEAA, 2 mM GlutaMAX, 25 ug/mL Insulin, 10 mM HEPES (Sigma, H4034), 60 ng/ml T3, 100 ng/ml Biotin, 1 μM cAMP and 25 ug/mL ascorbic acid (Sigma, A4403) for 45 days or longer.

Generation and maintenance of immunodeficient CD mice. All animal housing conditions and surgical procedures were approved by and conducted according to the Institutional Animal Care and Use Committee of City of Hope. ASPA^{nur7}/+(ASPA^{nur7}/J, 008607) and Rag2−/− mice (B6(Cg)-Rag2^{tm1.1Cgn}/J, 008449) were purchased from the Jackson Laboratory. ASPA^{nur7/+} mice were backcrossed with Rag2−/− mice for four generations and screened for homozygosity of ASP^{Anur7/nur7} and Rag2^{-/-} mutations.

Stereotaxic transplantation. Neonatal mice (P2-P4) were anesthetized on ice for 4 min and then placed onto a stereotaxic device. Cell suspension was injected into neonatal mouse brains using a Hamiliton syringe with 33 gauge needle. The following coordinates, which were modified from a published study42, were used for transplantation. The thalamus: (−0.5, 1, −2.5), the cerebellum: (−2.0, 0.8, −2.5), and the brain stem (−2.0, 0.8, −3.2). All the coordinates are (A, L, V) with reference to Lambda. A stands for anteroposterior from midline, L stands for lateral from midline, and V stands for ventral from the surface of brain, respectively. NPCs within 14 passages were transplanted bilaterally into the thalamus, and the cerebellar white matter and the brain stem at 200,000 cells per site in 2 uL and six sites per mouse.

ASPA enzymatic activity assay. The ASPA enzymatic assay was developed based on a published protocol43. Forty microliter of cell lysates or brain tissue protein supernatants was added to 10 μl of assay buffer, containing 250 mM Tris-HCl, pH 8.0, 250 mM NaCl, 2.5 mM DTT, 0.25% non-ionic detergent, 5 mM CaCl₂), 5 mM NAA (Sigma, 00920). The reaction mixture was incubated at 37ºC for 90 min, then the reaction was stopped by incubating the tubes in boiling water for 3 min. Reaction blank was created by adding 40 μl H₂O instead of protein homogenate. The reaction mixture was centrifuged at 13,000 rpm for 10 min to remove the precipitates. The supernatant was added into the enzyme assay buffer containing 50 mM Tris-HCl, pH 8.0, 50 mM NaCl, 2 mM a-ketoglutarate, 0.15 mM β-NADH, 1 mg/ml BSA and 10 units each of malate dehydrogenase and Glutamate-Oxaloacetate Transaminase. The reaction was incubated at RT for 20 min. The supernatant was transferred to a clear 96-well flat bottom plate and absorbance was measured at 340 nm using a plate reader.

NAA level measurement. Aqueous metabolites were extracted from the thalamus, brainstem and cerebellum of indicated mice using the method of perchloric acid (PCA, Sigma, 244253) as described44. Briefly, tissues were rapidly chopped into small pieces and collected into 1.5 ml Eppendorf tubes. 5 ml/g (wet weight basis) of 6% ice-cold PCA was added into each tubes, followed by vortexing for 30 s and incubating the samples on ice for additional 10 min. The mixture was centrifuged at 12,000 g for 10 min at 4ºC. The PCA supernatants were transferred into new tubes and neutralized with 2 M K2CO3, and placed on ice with lids open to allow CO2 to escape. Each sample was vortexed and incubated on ice for 30 min to precipitate the potassium perchlorate salt. The supernatant pH was adjusted to 7.4 +0.2, then centrifuged at 12,000 g for 10 min at 4° C. The supernatant was transferred to Eppendorf tubes and frozen on dry ice. The samples were then subjected to NMR analysis at the NMR Core Facility of City of Hope.

Electron microscopy (EM). Mice were deeply anesthetized with isoflurane, and perfused with 0.9% saline followed by 0.1 M Millonig's buffer containing 4% paraformaldehyde (PFA) and 2.5% glutaraldehyde at 37° C. Brain tissues were dissected and post-fixed in the same fixative overnight. A heavy metal staining protocol developed by Dr. Mark Ellisman's group45 was followed. Target tissues were cut into ˜150 μm vibratome sections using a Leica VT 1000S vibratome. The vibratome sections were then fixed overnight in 0.15 M cacodylate buffer, pH 7.4, containing 2.5% glutaraldehyde and 2 mM calcium chloride. The next day tissue sections were washed 5×3 minutes in 0.15 M cacodylate buffer, pH7.4, containing 2 mM calcium chloride, and then fixed in 0.15 M cacodylate buffer, pH7.4, containing 1.5% potassium ferrocyanide, 2% aqueous osmium tetroxide, and 2 mM calcium chloride for 1 hr. The samples were then placed in 1% thiocarbohydrazide (Acros Organics) for 20 min, followed by fixing in 2% osmium tetroxide for 30 min. The samples were then placed in 1% aqueous uranyl acetate solution at 4° C. for overnight. After washing with water 5×3 minutes, the samples were stained en bloc with Walton's lead aspartate in a 60ºC oven for 30 min. After another 5×3 minutes rinse in water, the samples were dehydrated and embedded in Durcupan ACM resin (Electron Microscopy Sciences). 70 nm-thick ultra-thin sections were cut using a Leica Ultracut UCT ultramicrotome with a diamond knife, picked up onto 200 mesh copper EM grids. Transmission electron microscopy was performed on an FEI Tecnai 12 transmission electron microscope equipped with a Gatan Ultrascan 2K CCD camera at the EM Core Facility of City of Hope.

Rotarod test. The motor performance of mice was tested by rotarod treadmill (Columbus Instruments) as described34. One-month-old or 3-month-old Aspa^{nur 7/nur7} Rag2−/− mice (CD mice) transplanted with indicated cells were assessed. Mice were tested for the latency on the rod when the rod was rotating at the accelerating speed (5−65 rpm) in a 5 min trial session. Each mouse was monitored for the latency 3 times per test.

Hanging wire test. Paw strength was assessed as indicator of neuromuscular function through a four-paw “hanging wire” approach as described46. Tape was put on a wire cage lid to form a 10 cmx15.5 cm field, in which mice were placed. After mice strongly gripped the wires, the lid was gently turned upside down and held approximately 20 cm above cushioned ground. The latency for mice to fall was measured. Wild type mice can usually hold onto the lid for at least 60 sec, therefore 60 sec was set as the cut-off latency. Neuromuscular disabilities would result in premature fall from the lid.

RNA preparation and RT-PCR analysis. Total RNAs were extracted from cells using the TRIazol reagent (Invitrogen, 15596018). Reverse transcription was performed with 1 ug of RNA using the Tetro cDNA synthesis kit (Bioline, BIO-65043). The primers used for PCR are listed in Table 2.

TABLE 2
Sequences of the Primers

| | | |
|---|---|---|
| Primer | Orien- | |
| name | tation | Sequence |

Primers for ASPA Exon Sequencing

| | | |
|---|---|---|
| ASPA-Exon1 | Forward | 5′-CTCCACTCAAGGGAATTCTGT-3′ |
| | Reverse | 5′-ACTGCATGTACGGACATGAA-3′ |
| ASPA-Exon2 | Forward | 5′-AGATTTGGCGACTGGTTCT-3′ |
| | Reverse | 5′-TGCACCTTCCCTCATAACTG-3′ |
| ASPA-Exon3 | Forward | 5′-ACTCTGTTGAAGCAAAGAGA-3′ |
| | Reverse | 5′-CAGAGCAAGACTCTGTCTCA-3′ |
| ASPA-Exon4 | Forward | 5′-TTCCATGATGCTACATGGTT-3′ |
| | Reverse | 5′-GCAAATCTGACCCAGGTTCCA-3′ |
| ASPA-Exon5 | Forward | 5′-TGTTCTCGAACTCCTGACCT-3′ |
| | Reverse | 5′-GCGAAGTGCTGTATGAGCTA-3′ |
| ASPA-Exon6 | Forward | 5′-GATCAAGACTGGAAACCAC-3′ |
| | Reverse | 5′-GAAGTGTAGTAAGGCAAAGC-3′ |

Primers for RT-PCR

| | | |
|---|---|---|
| Endo-OCT4 | Forward | 5′-CCTCACTTCACTGCACTGTA-3′ |
| | Reverse | 5′-CAGGTTTTCTTTCCCTAGCT-3′ |
| Endo-SOX2 | Forward | 5′-CCCAGCAGACTTCACATGT-3′ |
| | Reverse | 5′-CCTCCCATTTCCCTCGTTTT-3′ |
| Endo- | Forward | 5′-GAATCTTCACCTATGCCTGTG-3′ |
| NANOG | Reverse | 5′-ATCATTGAGTACACACAGCTG-3′ |
| Exo-OCT4 | Forward | 5′-CCTCACTTCACTGCACTGTA-3′ |
| | Reverse | 5′-TTATCGTCGACCACTGTGCTGCTG-3′ |
| Exo-SOX2 | Forward | 5′-CCCAGCAGACTTCACATGT-3′ |
| | Reverse | 5′-TTATCGTCGACCACTGTGCTGCTG-3′ |
| Exo-KLF4 | Forward | 5′-GATGAACTGACCAGGCACTA-3′ |
| | Reverse | 5′-TTATCGTCGACCACTGTGCTGCTG-3′ |
| Exo-cMYC | Forward | 5′-GCCACAGCATACATCCTGTC-3′ |
| | Reverse | 5′-TTATCGTCGACCACTGTGCTGCTG-3′ |
| Episomal- | Forward | 5′-CTCTAGAGCCTCTGCTAACCA-3′ |
| Exo-OCT4 | Reverse | 5′-TGTGCATAGTCGCTGCTTGAT-3′ |
| Episomal- | Forward | 5′-GCTCCCATCTTTCTCCACGTT-3′ |
| Exo-KLF4 | Reverse | 5′-GAAGCTTGAATTCCTGCAGGCA-3′ |
| Episomal- | Forward | 5′-AGAGCATCAGCCATATGGTAG-3′ |
| Exo-LIN28 | Reverse | 5′-GAAGCTTGAATTCCTGCAGGCA-3′ |
| Episomal- | Forward | 5′-CTCTAGAGCCTCTGCTAACCA-3′ |
| Exo-L-MYC | Reverse | 5′-TCGAATTTCTTCCAGATGTCC-3′ |
| ASPA | Forward | 5′-CGGAATTCATGACTTCTTGTCAC-3′ |
| | Reverse | 5′-GGACTAGTCTAATGTAAACAGCAG-3′ |
| hASPA | Forward | 5′-GATCAAGACTGGAAACCAC-3′ |
| | Reverse | 5′-GCGGCCTCATTCACAAACAC-3′ |
| hMBP | Forward | 5′-CTATAAATCGGCTCACAAGG-3′ |
| | Reverse | 5′-AGGCGGTTATATTAAGAAGC-3′ |
| mPLP | Forward | 5′-CACTTACAACTTCGCCGTCCT-3′ |
| | Reverse | 5′-GGGAGTTTCTATGGGAGCTCAGA-3′ |
| hOLIG2 | Forward | 5′-TGCGCAAGCTTTCCAAGAT-3′ |
| | Reverse | 5′-CAGCGAGTTGGTGAGCATGA-3′ |
| hNKX2.2 | Forward | 5′-GACAACTGGTGGCAGATTTCGCTT-3′ |
| | Reverse | 5′-AGCCACAAAGAAAGGAGTTGGACC-3′ |
| hSOX10 | Forward | 5′-CCACGAGGTAATGTCCAACATG-3′ |
| | Reverse | 5′-CATTGGGCGGCAGGTACT-3′ |

Immunocytochemistry. Cells were fixed with 4% PFA at RT for 5−10 minutes. After fixation, cells were washed with PBS twice and blocked with PBS with 0.1% triton (PBST) for 20 min. The fixed cells were incubated with primary antibodies at RT for 1 hour or at 4ºC for overnight, washed with PBS twice, then incubated with secondary antibodies at RT for 1 hour and washed. Primary antibodies used are listed in Table 3.

TABLE 3
List of Primary Antibodies

| Antigen | Dilution | Host Species | Cat. No. | Vendor |
|---|---|---|---|---|
| Sox1 | 1:1000 | Goat | AF3369 | R&D |
| Sox2 | 1:1000 | Goat | AF2018 | R&D |
| Pax6 | 1:500 | Rabbit | PRB-278P | Covance |
| N-cad | 1:1000 | Mouse | 610920 | BD Transduction |
| | | | | Laboratories ™ |
| Nestin | 1:1000 | Mouse | 611659 | BD Transduction |
| | | | | Laboratories ™ |
| Olig2 | 1:300 | Rabbit | GTX62440 | Genetex |
| hNA | 1:200 | Mouse | Ab191181 | Abcam |
| GFAP | 1:500 | Rabbit | Z0334 | Dako |
| MBP | 1:500 | Rat | Ab7349 | Abcam |
| NeuN | 1:200 | Rabbit | GTX16208 | Genetex |
| Tra-1-60 | 1:500 | Mouse | sc-21705 | Santa Cruz |
| Tra-1-81 | 1:500 | Mouse | sc-21706 | Santa Cruz |
| SSEA4 | 1:500 | Mouse | sc-21704 | Santa Cruz |
| Oct3/4 | 1:500 | Mouse | sc-5279 | Santa Cruz |
| Nanog | 1:500 | Rabbit | sc-33760 | Santa Cruz |
| Sox17 | 1:500 | Mouse | Ab84990 | Abcam |
| SMA | 1:500 | Rabbit | Ab5694 | Abcam |
| Tuj1 | 1:6000 | Rabbit | PRB-435P | Covance |

Immunohistochemistry. Immunohistochemistry was performed on paraformaldehyde (PFA)-fixed tissue. Animals were deeply anesthetized and transcardially perfused with ice-cold 0.9% saline followed by 4% PFA. Perfused brains were removed and post-fixed in 4% PFA overnight, then cryoprotected with 30% sucrose. Cryoprotected brains were flash frozen and stored at −80° C. For immunohistochemistry analysis, brain sections were permeabilized in PBS with PBST for 20 min, and washed 3×5 min in PBST. Sections were incubated with primary antibodies (Table 3) at 4 ºC for overnight. Following primary antibody incubation and washes, sections were incubated with secondary antibodies, including anti-mouse Cy3 (Jackson ImmunoResearch, 715−165−150), anti-rabbit Alexa Fluor 488 (Invitrogen, A21206), and anti-mouse Dylight 488 (Jackson ImmunoResearch, 715−487−003) at RT for 2 h, washed with 1×PBS, counter stained with Dapi, and mounted with the 4% PVA mounting medium. No antigen retrieval or detergents were required to optimize staining. Cell fate and proliferation status assessment were performed by double immunostaining using the anti-human nuclear antigen SC101, with antibodies against OLIG2, MAP2, GFAP, or Ki67. Confocal microscopy was performed on a Zeiss LSM 700 microscope (Zeiss), and the resulting images were analyzed with Zen 2.3 lite software (Zeiss). The entire brain was scanned. Z-stack imaging was performed to capture the full depth of staining for all the markers. The human nuclear antigen+ cells, the human nuclear antigen+OLIG2+ cells or human nuclear antigen+Ki67+ cells were counted. For quantification, slides in every eighth section from each mouse brain were selected. The orthogonal view tool was used to confirm co-staining for human nuclear antigen and MBP in the double-positive cells.

Vacuolation analysis A one-in-eight series of sections were stained with hematoxylin and eosin (H&E) and used to measure the volume of brain regions, including thalamus, cerebellum, and brain stem. The entire regions were scanned under a Zeiss confocal microscope. Z-stack images were acquired. The surface area of the vaculated brain regions and the intact brain regions was traced in Image J using images of the entire region. The volume was calculated by multiplying the sum of the surface area by the distance between sections sampled. % Vacuolation=the volume of vacuolated brain region/the volume of vacuolated brain region+the volume of intact brain region.

Tumor monitoring in ASPA1 NPC-transplanted mice. The ASPA1 NPCs were transplanted into CD mice for up to 10 months. Within the 10-month period, the ASPA1 NPC-transplanted CD mice were monitored monthly. Ten months after transplantation, the ASPA1 NPC-transplanted CD mice were perfused, sectioned and subjected to H&E staining or Ki67 staining.

Statistical analyses Data are shown as means+sd or means+se as specified in the figure legends. The number of mice per treatment group is indicated as “n” in the corresponding figure legends. No exclusion criteria were applied. Animals were assigned randomly to treatment groups. The study was not blinded. Student's t-test was used for statistical analysis as reported in each figure legend. p<0.05 was considered statistically significant.

### Example 2 Derivation and characterization of CD iPSCs

Primary dermal fibroblasts were obtained from three clinically affected Canavan disease patients (Table 4). Two Canavan disease patients (CD1 and CD2) each have heterozygous mutations at both nucleotide 527 (527G>A) and nucleotide 914 (914C>A) of the ASPA gene, resulting in a substitution of glycine by glutamic acid at codon 176 (G176E) and a substitution of alanine by glutamic acid at codon 305 (A305E). The third Canavan disease patient (CD3) has a homozygous mutation at nucleotide 854 of the ASPA gene (854A>C), resulting in a substitution of glutamic acid by alanine at codon 285 (E285A). E285A is the predominant mutation (accounting for over 82% of mutations) among the Ashkenazi Jewish population28,29 , whereas A305E is the most common mutation (60%) in non-Jewish Canavan disease patients30. G176E is a new ASPA mutation identified in Canavan disease patients as disclosed herein. Normal human fibroblast cells IMR90 were included as the wild type (WT) control (Table 4).

TABLE 4
Wild Type and CD Cells Used in the Study

| iPSCs | Fibroblast | | | Age at | ASPA | iPSC | Mycoplasma |
|---|---|---|---|---|---|---|---|
| lines | Catalog # | Vendor | Gender | biopsy | mutation | karyotype | status |
| WT | I90-10 | Coriell | Female | 16 FW | No | 46, XX | Negative |
| CD1 | GM00059 | Coriell | Female | 1 year | G176E, | 46, XX | Negative |
| | | | | | A305E | | |
| CD2 | GM00060 | Coriell | Male | 2 year | G176E, | 46, XY | Negative |
| | | | | | A305E | | |
| CD3 | GM04268 | Coriell | Male | 2 year | E285A | 46, XY | Negative |

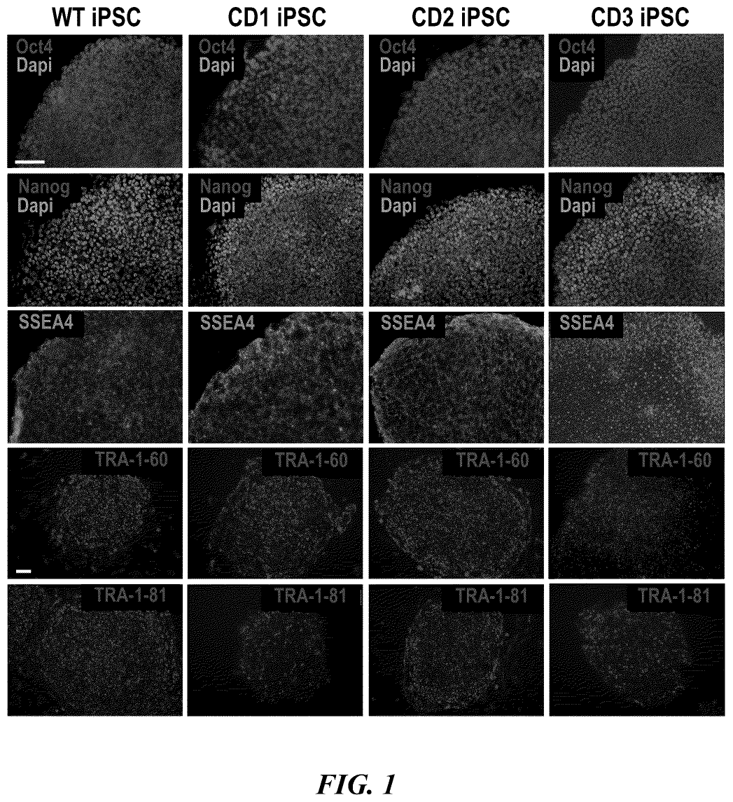

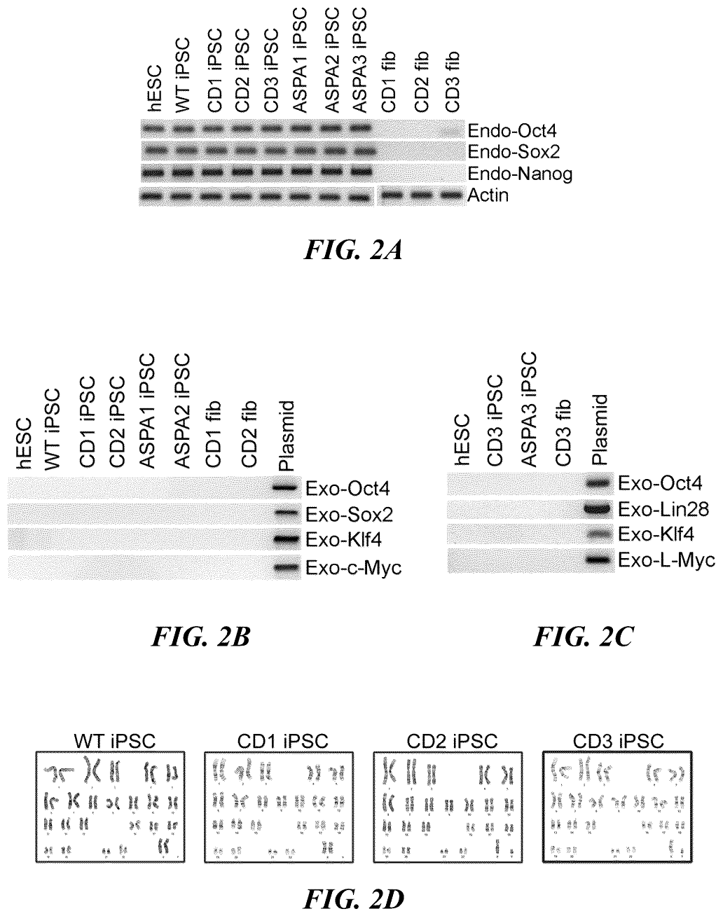

These fibroblasts were reprogrammed to generate WT and CD patient iPSCs (CD iPSCs) using the reprogramming factors, including OCT4, SOX2, KLF4, LIN28 and MYC, via episomal reprogramming31,32 or viral transduction1. The iPSC lines derived from both normal human fibroblasts and CD patient fibroblasts expressed the key human pluripotency genes, OCT4 and NANOG, and the human embryonic stem cell (ESC)-specific surface markers, SSEA4, TRA-1−60 and TRA-1-81 (FIG. 1). Activation of the endogenous OCT4, SOX2, and NANOG gene expression was observed in both WT and CD iPSCs as revealed by RT-PCR analysis (FIG. 2A). In contrast, the expression of the exogenous reprogramming factors, OCT4, SOX2, KLF4, LIN28, and MYC, was not detectable in these iPSCs (FIGS. 2B and 2C). Cytogenetic analysis confirmed normal karyotype in all iPSC clones tested (FIG. 2D).

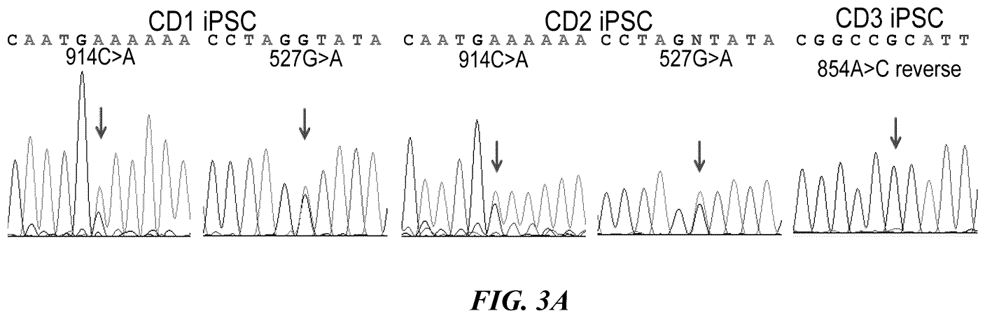

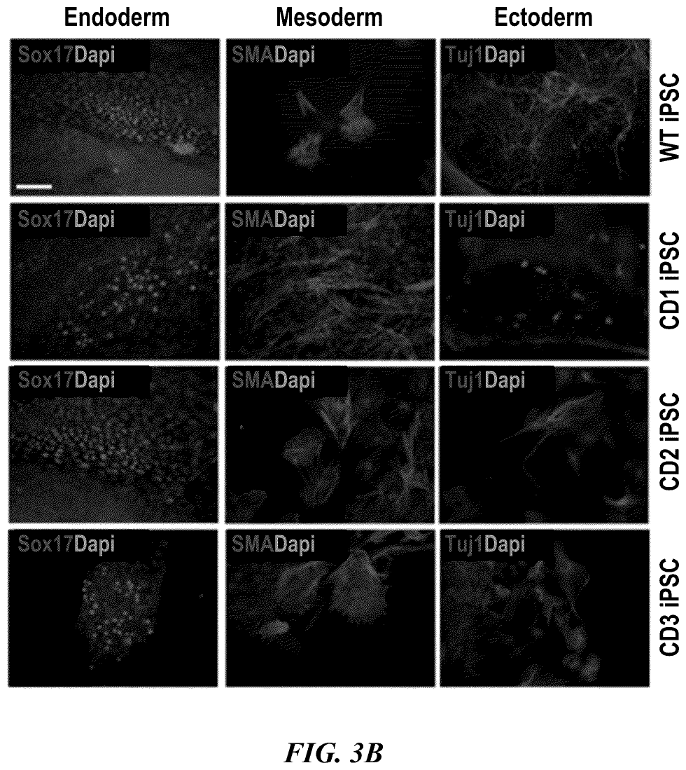

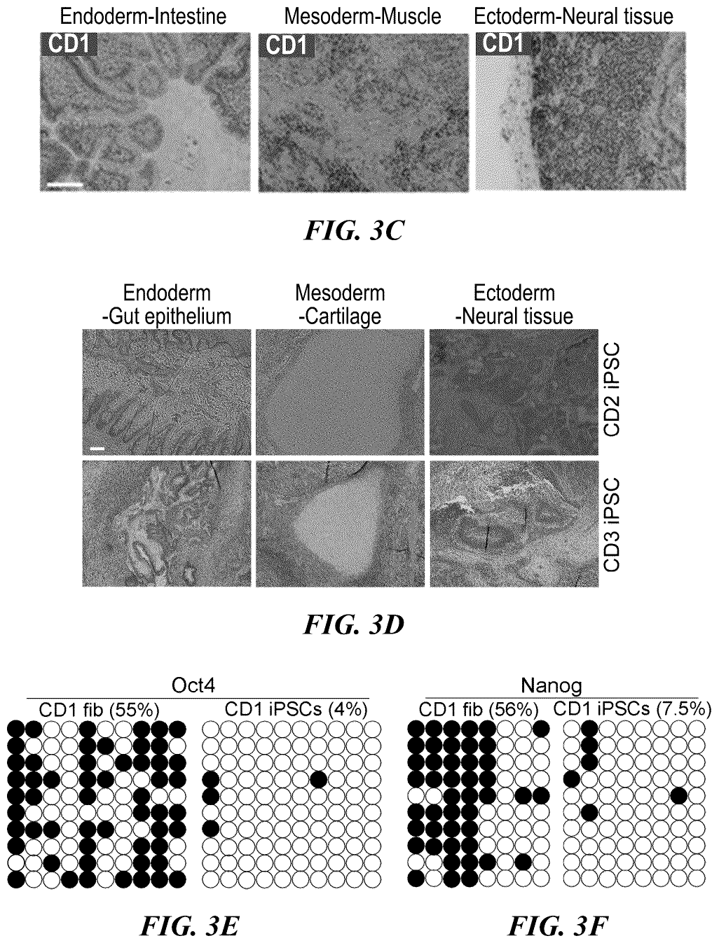

Sequence analysis confirmed that the CD patient 1 (CD1) and CD patient 2 (CD2) iPSCs contain two heterozygous mutations at nucleotide 527 (527G>A) and nucleotide 914 (914C>A) of the ASPA gene, whereas the CD patient 3 (CD3) iPSCs harbor a homozygous mutation at nucleotide 854 of the ASPA gene (854A>C) (FIG. 3A). Embryoid body (EB) formation assay was performed to demonstrate the pluripotent potential of the identified CD iPSC clones. Both WT and CD iPSCs could differentiate into characteristic SOX17-positive endodermal cells, smooth muscle actin (SMA)-positive mesodermal cells, and Blll tubulin (TUJ1)-positive ectoderm cells (FIG. 3B). The in vivo developmental potential of CD iPSCs was demonstrated by teratoma formation assay. The CD iPSCs were able to develop teratomas that contain tissues representing all three germ layers in transplanted immunodeficient NSG mice (FIGS. 3C and 3D).

Bisulfite sequencing analysis revealed that the endogenous Oct4 and Nanog promoters were largely demethylated in CD iPSCs. In contrast, the Oct4 and Nanog promoters in the parental CD fibroblast cells were highly methylated (FIGS. 3E and 3F). Together, these results demonstrate that we have successfully derived CD iPSCs that are characteristic pluripotent stem cells and contain patient ASPA mutations.

### Example 3 Generation of Genetically-Corrected ASPA iPSCs

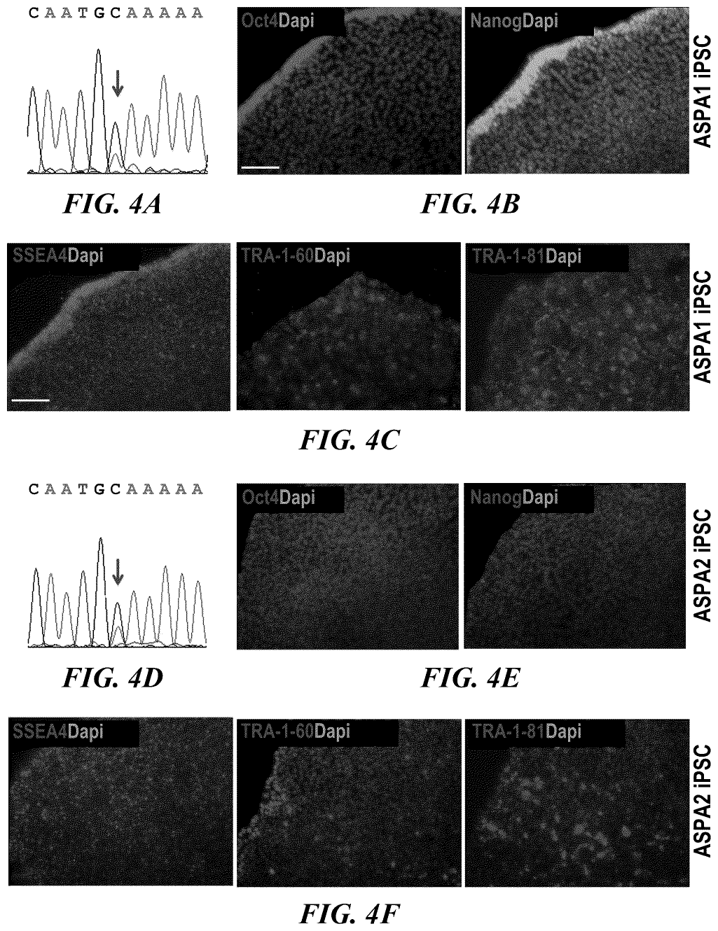

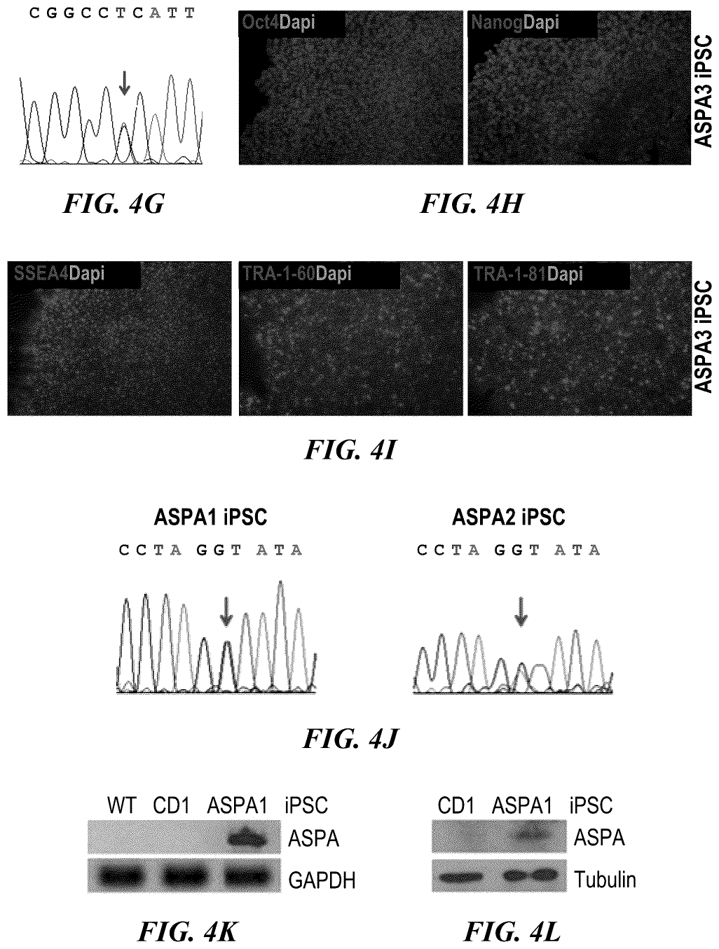

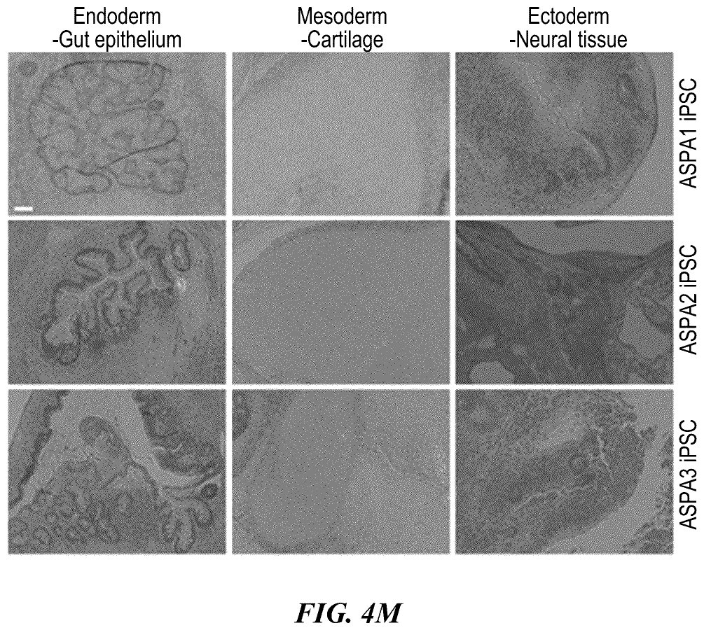

Because Canavan disease is caused by genetic mutations in the ASPA gene, in order to correct CD patient iPSCs, CD patient iPSCs were transduced with lentivirus expressing the human WT ASPA gene under the constitutive human EF1a promoter. The genetically corrected CD patient iPSCs were termed ASPA iPSCs. The ASPA1 (or ASPA-CD1), ASPA2 (or ASPA-CD2), and ASPA3 (or ASPA-CD3) iPSCs were derived from CD patient 1, CD patient 2, and CD patient 3 iPSCs, respectively. The presence of the WT ASPA gene sequence was confirmed in the ASPA1, ASPA2, and ASPA-3 iPSCs (FIGS. 4A, 4D, 4G and 4J).

Immunostaining revealed that the ASPA IPSCs continued to express the pluripotency factors OCT4 and NANOG and the human ESC surface markers SSEA4, TRA-1−60 and TRA-1−81 (FIGS. 4B, 4C, 4E, 4F, 4H, 4I). RT-PCR confirmed induction of the endogenous OCT4, SOX2 and NANOG expression in ASPA iPSCs (FIG. 2A). In contrast, the exogenous reprogramming factors, OCT4, SOX2, KLF4, LIN28, and MYC were not detectable in these iPSCs (FIGS. 2B and 2C). The ASPA iPSCs also maintained their developmental potential. After transplanting into immunodeficient NSG mice, the ASPA1, ASPA2, and ASPA3 iPSCs were able to develop teratomas that contain all three germ layers (FIG. 4M).

### Example 4 Neural Differentiation of ASPA iPSCs

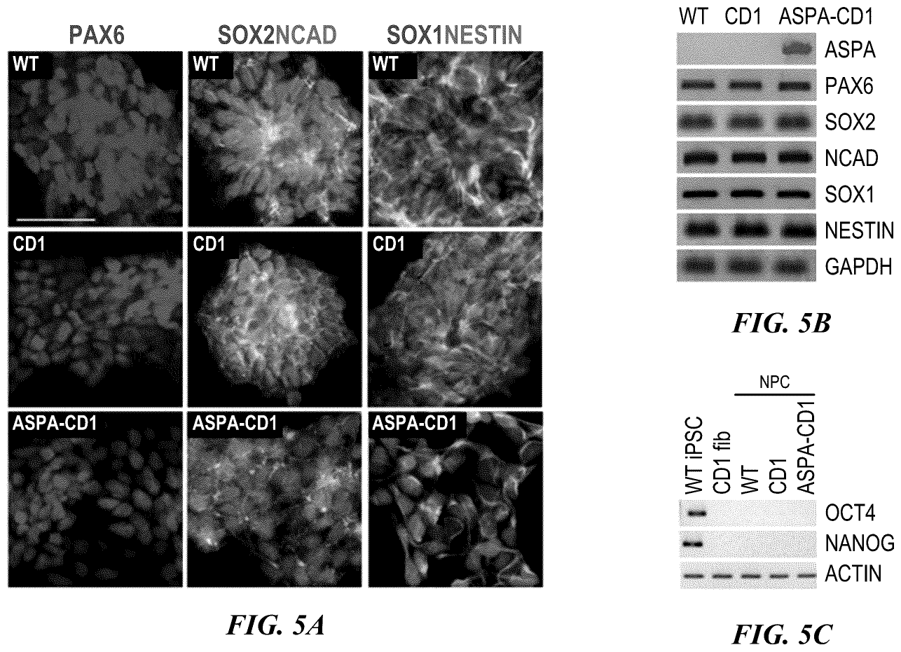

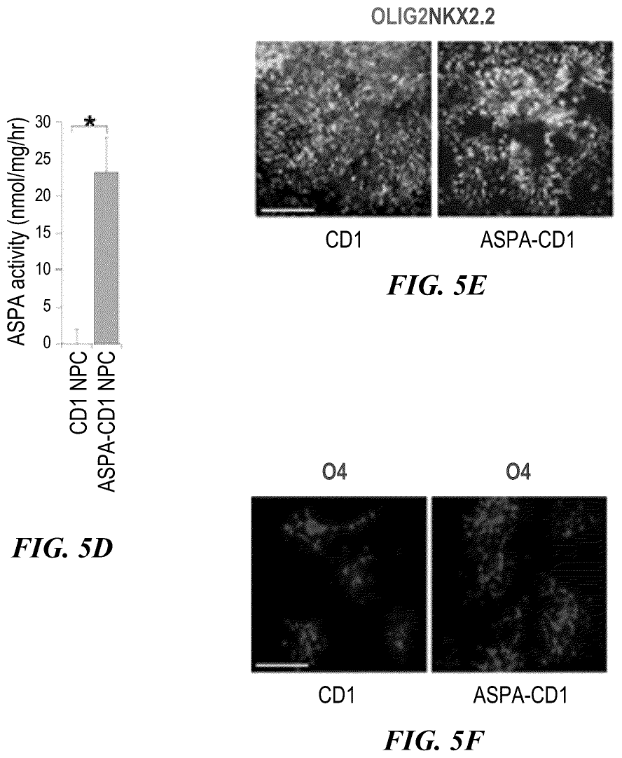

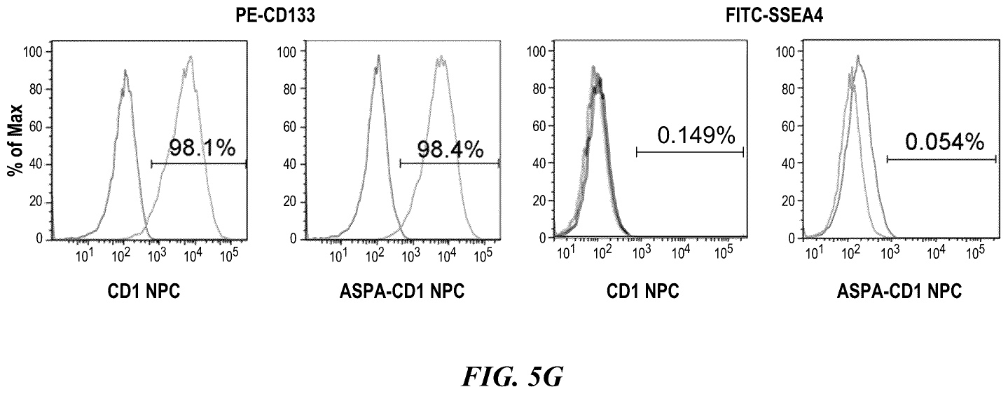

WT, CD1, and ASPA1 iPSCs were differentiated into neural progenitor cells (NPCs) following a published protocol33. The NPCs derived from all three iPSC lines expressed typical NPC markers, including PAX6, SOX2, N-cadherin, SOX1, and NESTIN (FIGS. 5A & 5B). In contrast, no expression of the pluripotency factors OCT4 and NANOG was detected in either type of NPCs (FIG. 5C). The ASPA1 iPSC-derived NPCs (ASPA1 NPCs) also expressed the ASPA gene (FIG. 5B).

Moreover, the ASPA1 NPCs exhibited potent ASPA enzymatic activity, compared to CD1 iPSC-derived NPCs (CD1 NPCs), which exhibited no detectable ASPA activity (FIG. 5D). Further differentiation of the ASPA1 NPCs along the oligodendroglial lineage allowed obtaining OLIG2+NKX2.2+pre-OPCs by day 13 of differentiation, and 04+OPCs by day 80 of differentiation (FIGS. 5E & 5F). Similar results were obtained from CD1 NPCs (FIGS. 5E & 5F). These results demonstrate that the ASPA1 NPCs not only possess potent ASPA enzymatic activity but also have the capacity to differentiate into oligodendroglial lineage cells.

Fluorescence-activated cell sorting (FACS) revealed that the vast majority of the CD1 NPCs and ASPA1 NPCs are CD133-positive NPCs, with minimal contamination of undifferentiated iPSCs as revealed by the negligible fraction of SSEA4-positive cells (FIG. 5G). Together, these results demonstrate the identity, purity, and potency of the ASPA1 NPCs.

### Example 5 the ASPA NPCs can Survive and Provide Functional Rescue in Transplanted CD Mice

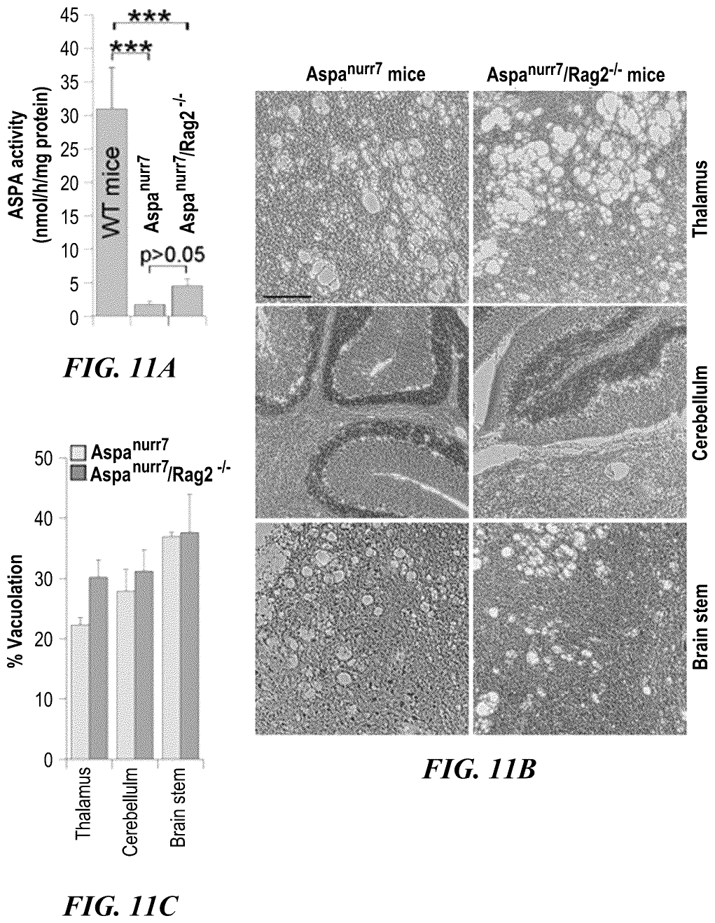

The Aspa^{nur} 7/nur7 mouse contains a nonsense mutation (Q193X) in the ASPA gene³⁴. Because Aspa^{nur}7/nur7 mice exhibit key pathological phenotypes resembling those of CD patients, including loss of ASPA enzymatic activity, elevated NAA levels, and extensive sponge degeneration in various brain regions34, it is considered an authentic animal model for CD. Therefore, the Aspa^{nur} 7/nur7 mouse provides an excellent platform for testing the therapeutic effects of NPCs derived from the genetically-corrected ASPA iPSCs. Because human cells need to be transplanted into a CD mouse model, an immunodeficient ASPA^{nur7/nur7} mouse model was generated by breeding the ASPA^{nur7/nur7} mice with immunodeficient Rag2 1- mice, which lack mature B and T lymphocytes. The resultant ASPAnur7/nur7/Rag2−/− mice are largely similar to the parental ASPA^{nur7/nur7} mice, both of which exhibited substantially reduced ASPA enzymatic activity in the brain, compared to the WT mice (FIG. 11A). Besides deficiency the ASPA activity, spongy degeneration as revealed by vacuolation is another characteristic feature of CD patients and mouse models. Extensive vacuolation was observed in various brain regions of both parental Aapa^{nur}7/nur7 and the Aapa^{nur7/nur7} /Rag2−/− mice, including thalamus, cerebellum and brain stem (FIGS. 11B & C). These results together indicate that the Aapa^{nur7/nur7} /Rag2^{-/-} mice, which are called immunodeficient CD mice or CD mice for short, exhibit typical CD phenotypes, similar to the parental Aapanur7/nur7 mice. These CD mice were used for transplantation to test the effect of the ASPA-CD NPCs in the following.

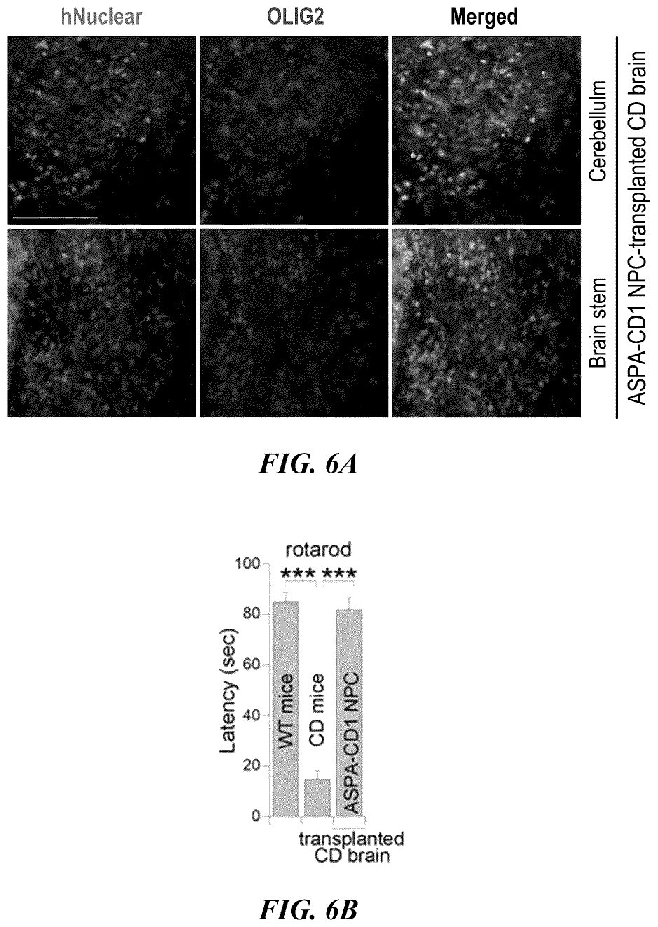

The ASPA1 NPCs differentiated from the ASPA1 iPSCs were transplanted into brains of neonatal CD mice. Two-hundred thousand cells in 2 uL volume were injected stereotactically into 6 sites of the brain of neonatal CD mice (see Methods). One month after transplantation, mouse brains were harvested and analyzed by immunostaining for human nuclear antigen to identify the transplanted human cells, and for OLIG2, a marker for oligodendroglial lineage cells. The transplanted ASPA1 NPCs were able to survive and express OLIG2 in examined brain regions, including cerebellum and brain stem (FIG. 6A). Moreover, the CD mice that had received ASPA1 NPCs demonstrated substantially improved rotarod performance, compared to CD mice without transplantation (FIG. 6B). These results indicate that the ASPA-expressing NPCs can survive in brains of CD mice, differentiate into oligodendroglial lineage cells and improve the motor function of CD mice.

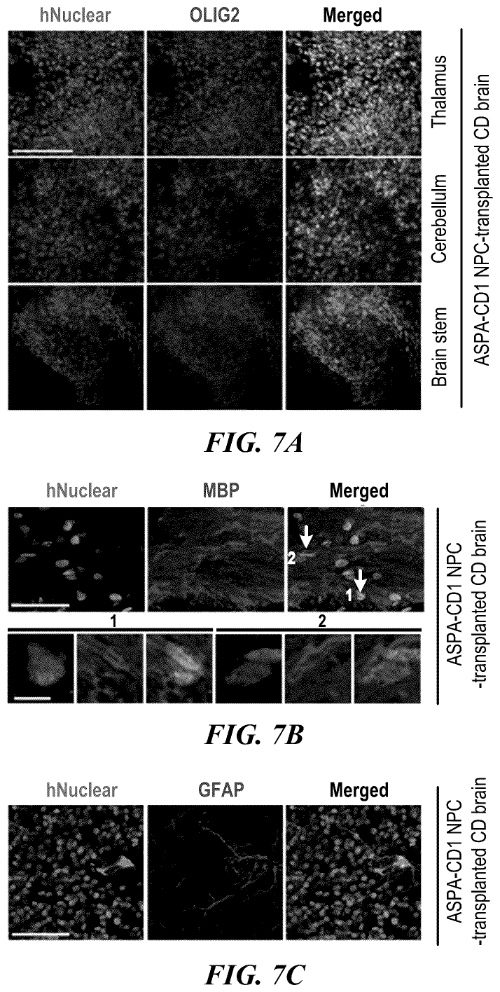

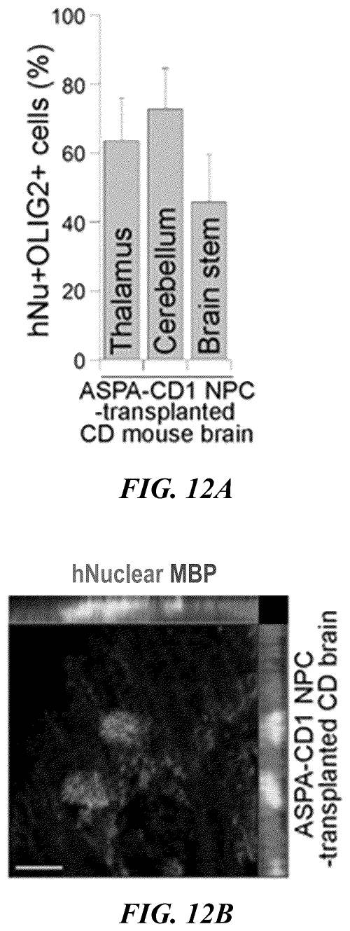

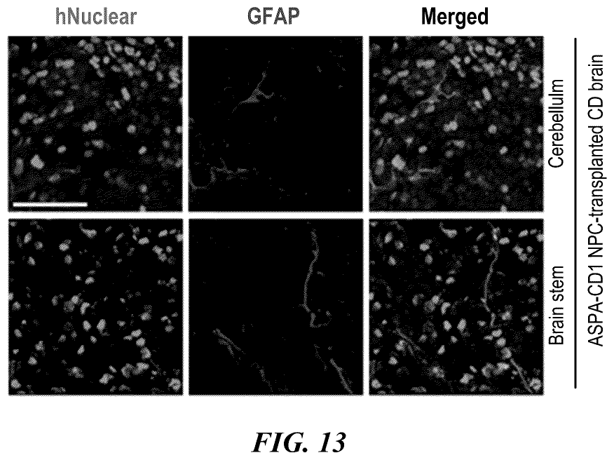

In another set of experiments, the ASPA1 NPCs were transplanted into brains of neonatal CD mice and the mice were allowed to survive for 3 months. Brains of the transplanted mice were then harvested and analyzed by co-staining for human nuclear antigen and the oligodendrocyte lineage transcription factor OLIG2. The transplanted ASPA NPCs were able to survive three months after transplantation and differentiate into oligodendroglial lineage cells (FIG. 7A). Quantification of the human nuclear antigen-positive and OLIG2-positive cells revealed that more than 60% of the human engrafted cells had differentiated into OLIG2+ cells in the thalamus of CD mice, about 72% and 45% of human cells became OLIG2+ cells in the cerebellum and brain stem, respectively (FIG. 12A). Furthermore, confocal microscopy analysis revealed that the engrafted human cells also differentiated into mature oligodendrocytes that expressed myelin basic protein (MBP) in the ASPA1 NPC-transplanted CD mouse brains (FIG. 7B). The co-staining for human nuclear antigen and MBP in the grafted cells was confirmed by orthogonal view of the confocal images (FIG. 12B). A fraction of the engrafted human cells differentiated into GFAP+astrocytes (FIGS. 7C & 13) These results indicate that the ASPA-expressing NPCs can survive long-term transplantation and give rise to oligodendroglial lineage cells in the transplanted brains.

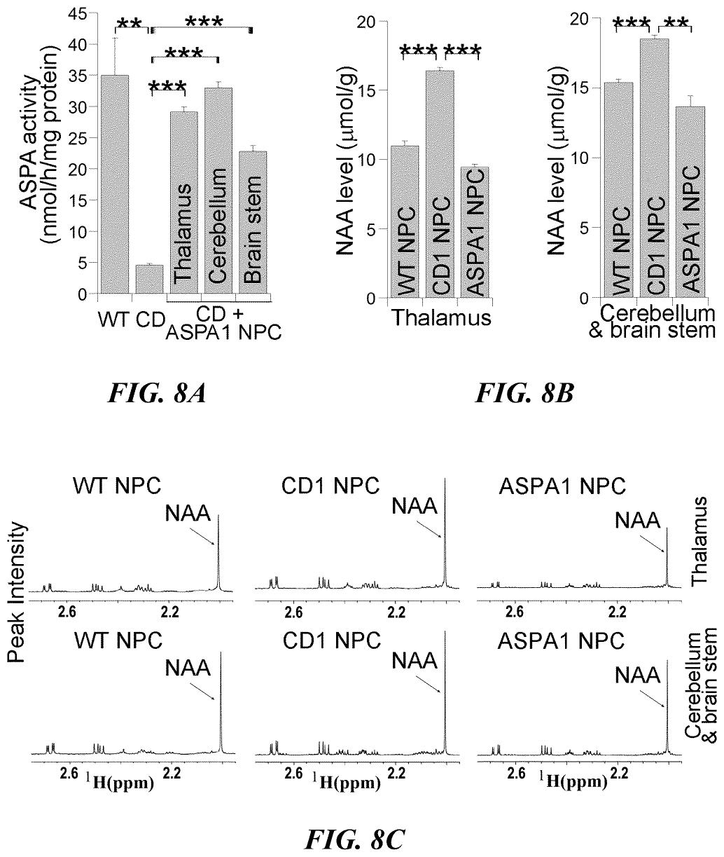

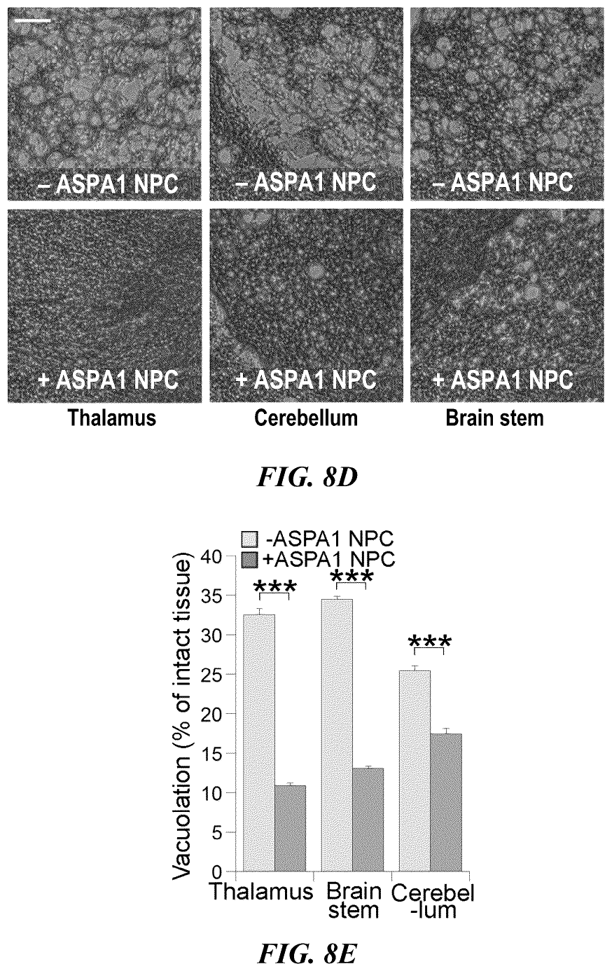

Because the deficiency in ASPA enzymatic activity is the underlying cause of disease phenotypes in both CD patients and animal models, the ASPA enzymatic activity was determined in CD mouse brains harvested three months after ASPA1 NPC transplantation. Potent ASPA enzymatic activity was detected in various brain regions of the ASPA1 NPC-transplanted mice, including thalamus, cerebellum and brain stem, compared to that in CD mouse brains without transplantation (FIG. 8A).

It has been shown that ASPA deficiency leads to elevated NAA levels in brains of both CD patients and mouse models 15,34−36. Consistent with elevated ASPA enzymatic activity, reduced NAA level in the ASPA1 NPC-transplanted CD mouse brains, compared to that in CD1 NPC-transplanted CD mouse brains, was detected (FIGS. 8B, 8C). These results indicate that transplantation of the ASPA1 NPCs was able to rescue the deficiency of ASPA enzymatic activity and reduce NAA levels, the major defects in both CD patients and mouse models.

Extensive spongy degeneration is another key pathological feature of CD patients and mouse models, which is revealed by vacuolation in various brain regions 15,34−36. Consistent with the observation of elevated ASPA enzymatic activity and reduced NAA levels in the brains of the ASPA1 NPC-transplanted CD mice, substantially reduced extent of vacuolation was detected in various brain regions of the ASPA1 NPC-transplanted CD mice, including thalamus, cerebellum and brain stem (FIGS. 8D, 8E).

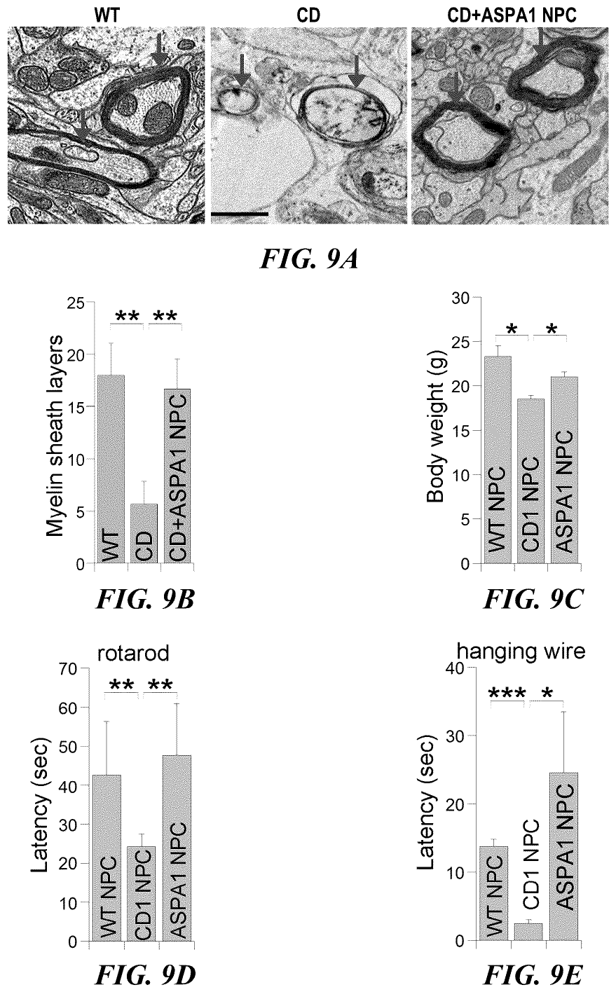

It has been suggested that vacuolation results from myelin destruction in brains of CD mice34. Consistent with extensive vacuolation in the brains of CD mice, substantially reduced thickness of myelin sheaths was observed in the brains of CD mice, compared to that from WT mice (FIGS. 9A, 9B). The myelin sheaths in the ASPA1 NPC-transplanted CD brains were much thicker than that of untreated CD brains, but resembled more to that of WT brains (FIGS. 9A, 9B). These results further support the therapeutic potential of the ASPA1 NPCs for their ability to ameliorate the pathological phenotypes of CD.

In addition to improvement of CD phenotypes at cellular level, the transplantation of the ASPA1 NPCs was also associated with systemic effect on CD mice. Weight loss has been reported in CD mice22,26,35. Three months after transplantation, a substantial increase of body weight was detected in CD mice transplanted with the ASPA1 NPCs or WT NPCs, compared to that in CD mice transplanted with the CD1 NPCs (FIG. 9C).

Defect in motor performance is typical of CD patients and animal models ^{15,34-36}. To determine if transplantation of the ASPA1 NPCs can rescue the defective motor performance in CD mice, CD mice transplanted with the WT NPCs, CD1 NPCs, or ASPA1 NPCs were tested in two motor skill behavioral paradigms. Three months after transplantation, CD mice that had received intracerebral injection of various NPCs were examined on the accelerating rotarod treadmill, which is designed for testing motor coordination and balance. Both the WT NPCs and the ASPA1 NPCs improved rotarod performance in transplanted CD mice substantially, compared to the CD1 NPCs, whereas no significant difference between the mice treated with the WT NPCs or the ASPA1 NPCs were detected (FIG. 9D). A hanging wire approach was used to evaluate paw strength as an indication of neuromuscular function37. Substantial enhancement of the grip strength was detected in a hanging wire test in both the WT NPCs and the ASPA1 NPC-transplanted CD mice, compared to that in the CD1 NPC-transplanted CD mice (FIG. 9E). These results together indicate that transplantation with the ASPA1 NPCs can improve motor functions in CD mice.

### Example 6 the ASPA2 NPCs and ASPA3 NPCs can Rescue Disease Phenotypes in CD Mice

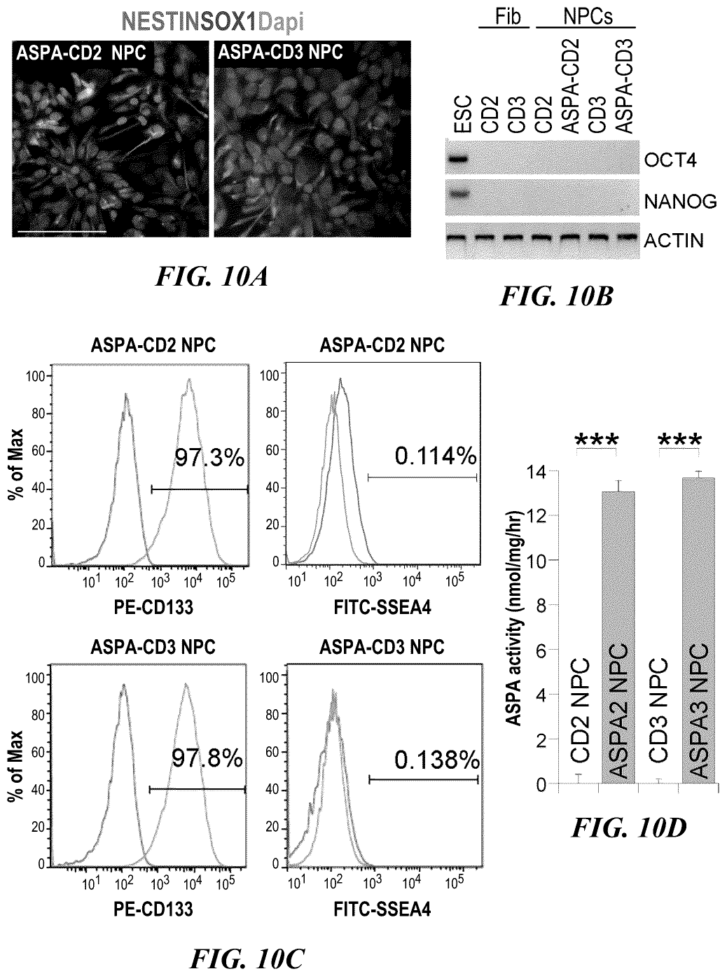

Having observed robust improvement of disease phenotype in CD mice transplanted with the ASPA1 NPCs, the effect of ASPA NPCs derived from CD patient 2 and CD patient 3 iPSCs were tested. CD2 iPSCs and CD patient 3 iPSCs were transduced with the ASPA-expressing lentivirus, and then these iPSCs were differentiated into NPCs. The resultant WT ASPA-expressing NPCs were termed the ASPA2 NPCs and ASPA3 NPCs, respectively. Both the ASPA2 NPCs and ASPA3 NPCs expressed typical NPC markers NESTIN and SOX1 (FIG. 10A). In contrast, no expression of the pluripotency factors OCT4 and NANOG was detected in the ASPA2 NPCs and ASPA3 NPCs (FIG. 10B).

The ASPA2 NPCs and ASPA3 NPCs were sorted by positive selection using the NPC surface marker CD133 and by negative selection using the human ESC surface marker SSEA4. The vast majority of the differentiated cells were CD133-positive and SSEA4-negative (FIG. 10C). The CD133-positive and SSEA4-negative cell population was harvested for transplantation experiments. Before transplantation, ASPA activity in the ASPA2 and ASPA3 NPCs was tested, and it was found that both the ASPA2 NPCs and ASPA3 NPCs exhibited potent ASPA activity compared to CD2 NPCs and CD3 NPCs (FIG. 10D). In summary, the identity, purity and potency of the ASPA2 NPCs and ASPA3 NPCs before transplantation have been demonstrated.

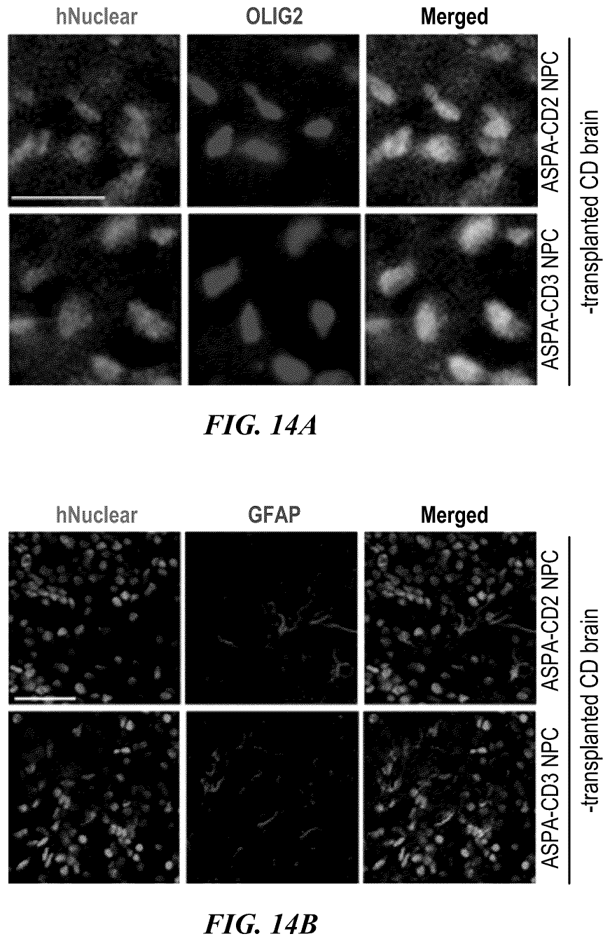

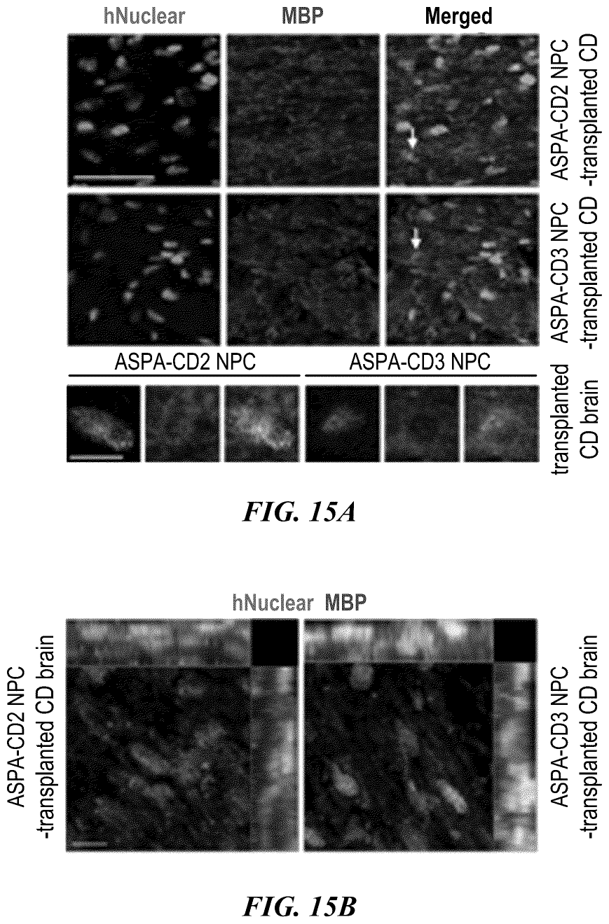

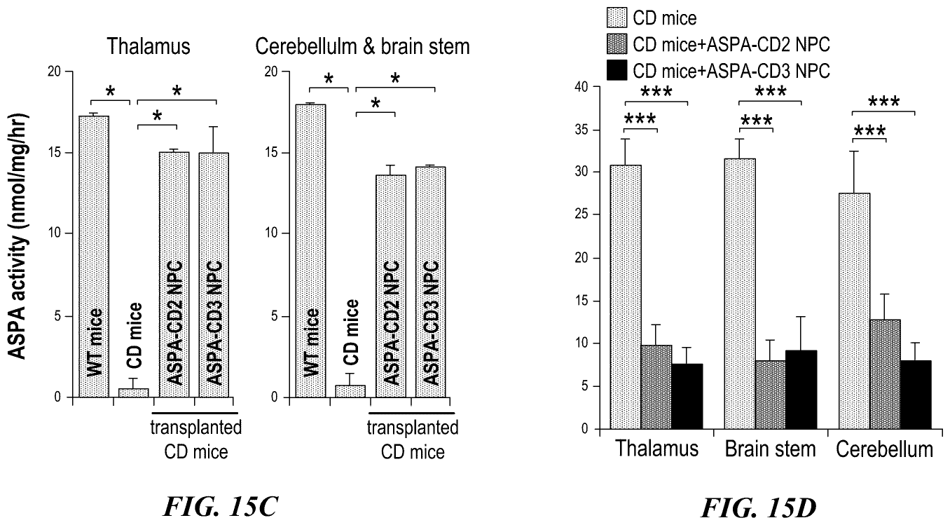

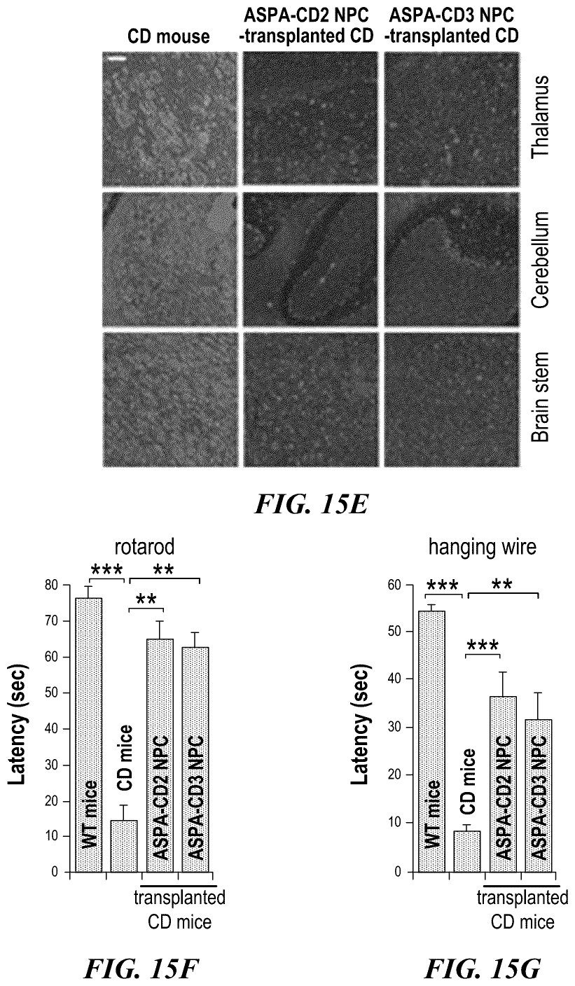

The sorted CD133-positive and SSEA4-negative ASPA2 NPCs and ASPA3 NPCs were transplanted into brains of neonatal CD mice, as described above and in Methods. Three months after transplantation, cells that were positive for both human nuclear antigen and OLIG2 in the transplanted CD mouse brains were detected (FIG. 14A). Moreover, the engrafted cells were able to give rise to MBP-positive mature oligodendrocytes (FIG. 15A). The presence of grafted cells that are positive for both human nuclear antigen and MBP in the transplanted brains was confirmed by orthogonal view of the confocal images (FIG. 15B). A portion of the transplanted cells gave rise to GFAP-positive astrocytes in the ASPA2 NPC or ASPA3 NPC-tansplanted CD brains (FIG. 14B).

ASPA enzymatic activity in the brains of CD mice transplanted with the ASPA2 NPCs or ASPA3 NPCs was examined. Potent ASPA enzymatic activity was detected in multiple brain regions of the transplanted brains, including the thalamus, cerebellum and brain stem, compared to that in CD mouse brains without transplantation (FIG. 15C). Similar to the ASPA1 NPC-transplanted CD mice, markedly reduced vacuolation was detected in the ASPA2 NPC or ASPA3 NPC-transplanted CD mouse brains, including the thalamus, cerebellum and brain stem (FIGS. 15D & 15E).

Next, CD mice received the ASPA2 NPCs or ASPA3 NPCs were tested on the accelerating rotarod treadmill. Both the ASPA2 NPCs and ASPA3 NPCs improved rotarod performance in the transplanted CD mice substantially, compared to CD mice without transplantion (FIG. 15F). Substantial enhancement of the grip strength was also detected in a hanging wire test in the CD mice transplanted with either the ASPA2 NPCs or ASPA3 NPCs, compared to that in CD mice without transplantation (FIG. 15G). These results together indicate that transplantation with either the ASPA2 NPCs or ASPA3 NPCs can improve motor functions in a mouse model of CD substantially. These results provide a proof-of-concept that NPCs derived from genetically corrected CD patient iPSCs have therapeutic potential to ameliorate the pathological phenotypes of CD.

### Example 7 No Tumor Formation in the ASPA1 NPC-Transplanted CD Mice

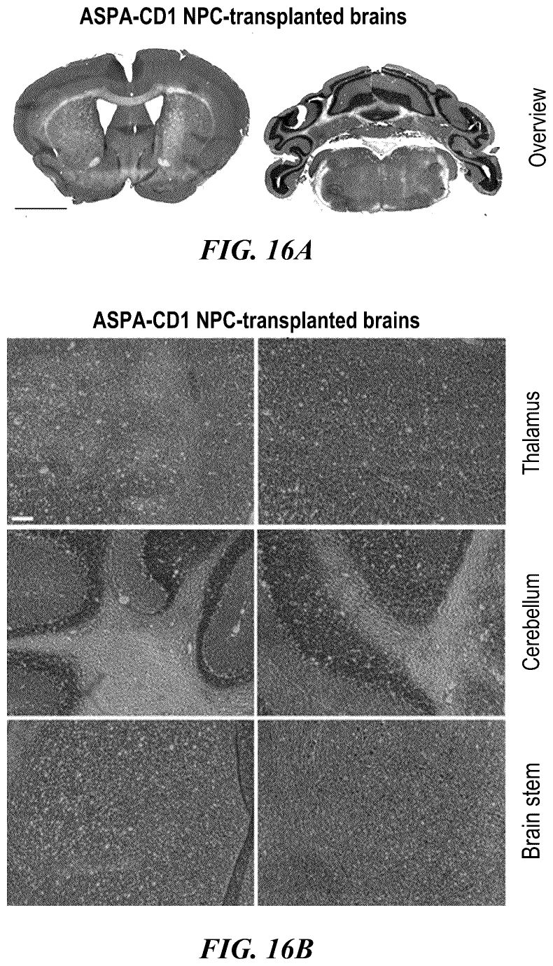

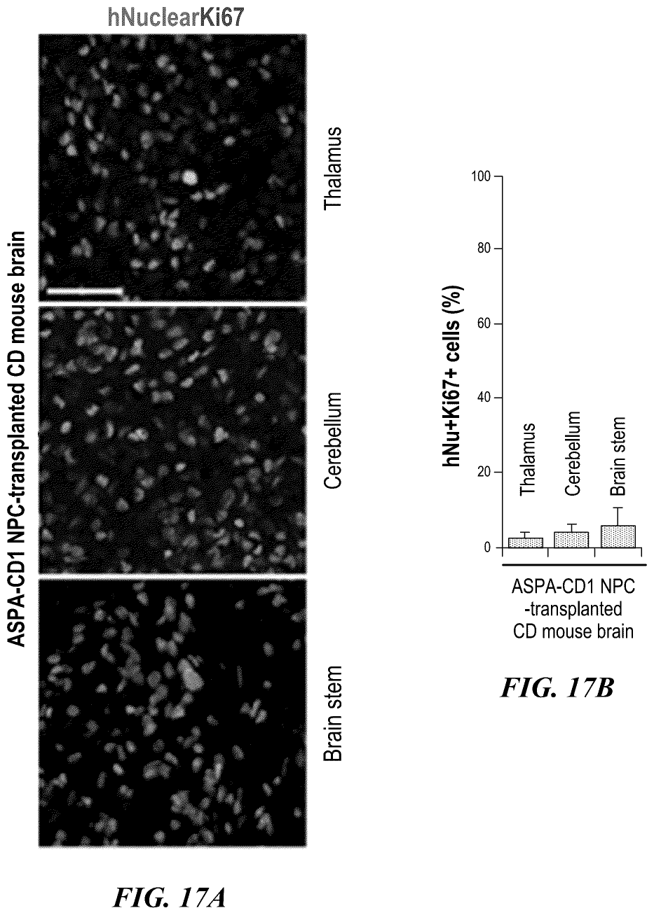

The ASPA1 NPCs were transplanted into the brains of CD mice for up to 10 months. Within these 10 months, the transplanted mice were monitored monthly and no sign of tumor formation was observed (Table 5). At the end of the 10th month, the transplanted mice were harvested and analyzed by H&E staining for further tumor analysis. No typical tumor tissue was found in these sections (FIGS. 16A and 16B). The lack of tumor formation in the ASPA1 NPC-transplanted mice was correlated with a very low mitotic index, as revealed by the low percentage of human nuclear antigen-positive and Ki67-positive cells in the ASPA1 NPC-grafted brains (FIGS. 17A and 17B).

TABLE 5
Safety Monitoring of ASPA1 NPC-Transplanted Mice
Monitoring Tumor Formation in ASPA1 NPC-transplanted CD mice

| | Month | | | | | | | | | |
|---|---|---|---|---|---|---|---|---|---|---|
| | 1^{st} | 2^{nd} | 3^{rd} | 4^{th} | 5^{th} | 6^{th} | 7^{th} | 8^{th} | 9^{th} | 10^{th} |
| ASPA1 | No | No | No | No | No | No | No | No | No | No |
| NPC- | tumor | tumor | tumor | tumor | tumor | tumor | tumor | tumor | tumor | tumor |
| transplanted | (n = 3) | (n = 3) | (n = 3) | (n = 3) | (n = 3) | (n = 3) | (n = 3) | (n = 3) | (n = 3) | (n = 3) |
| CD mice | | | | | | | | | | |

All publications and patent documents cited herein are incorporated by reference.

### REFERENCES

1 Takahashi, K., Tanabe, K., Ohnuki, M., Narita, M., Ichisaka, T., Tomoda, K., and Yamanaka, S. Induction of pluripotent stem cells from adult human fibroblasts by defined factors. Cell 131, 861−872 (2007).
2 Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., Slukvin, II, and Thomson, J. A. Induced pluripotent stem cell lines derived from human somatic cells. Science 318, 1917−1920 (2007).
3 Lowry, W. E., Richter, L., Yachechko, R., Pyle, A. D., Tchieu, J., Sridharan, R., Clark, A. T., and Plath, K. Generation of human induced pluripotent stem cells from dermal fibroblasts. Proc Natl Acad Sci USA 105, 2883−2888 (2008).
4 Park, I. H., Zhao, R., West, J. A., Yabuuchi, A., Huo, H., Ince, T. A., Lerou, P. H., Lensch, M. W., and Daley, G. Q. Reprogramming of human somatic cells to pluripotency with defined factors. Nature (2008).
5 Raya, A., Rodriguez-Piza, I., Guenechea, G., Vassena, R., Navarro, S., Barrero, M. J., Consiglio, A., Castella, M., Rio, P., Sleep, E., Gonzalez, F., Tiscornia, G., Garreta, E., Aasen, T., Veiga, A., Verma, I. M., Surralles, J., Bueren, J., and Izpisua Belmonte, J. C. Disease-corrected haematopoietic progenitors from Fanconi anaemia induced pluripotent stem cells. Nature 460, 53−59 (2009).
6 Hacein-Bey-Abina, S., Garrigue, A., Wang, G. P., Soulier, J., Lim, A., Morillon, E., Clappier, E., Caccavelli, L., Delabesse, E., Beldjord, K., Asnafi, V., Macintyre, E., Dal Cortivo, L., Radford, I., Brousse, N., Sigaux, F., Moshous, D., Hauer, J., Borkhardt, A., Belohradsky, B. H., Wintergerst, U., Velez, M. C., Leiva, L., Sorensen, R., Wulffraat, N., Blanche, S., Bushman, F. D., Fischer, A., and Cavazzana-Calvo, M. Insertional oncogenesis in 4 patients after retrovirus-mediated gene therapy of SCID-X1. J Clin Invest 118, 3132−3142 (2008).
7 Glaser, T., Schmandt, T., and Brustle, O. Generation and potential biomedical applications of embryonic stem cell-derived glial precursors. J Neurol Sci 265, 47−58 (2008).
8 Brustle, O., Jones, K. N., Learish, R. D., Karram, K., Choudhary, K., Wiestler, O. D., Duncan, I. D., and Mckay, R. D. Embryonic stem cell-derived glial precursors: a source of myelinating transplants. Science 285, 754−756 (1999).
9 Keirstead, H. S., Nistor, G., Bernal, G., Totoiu, M., Cloutier, F., Sharp, K., and Steward, O. Human embryonic stem cell-derived oligodendrocyte progenitor cell transplants remyelinate and restore locomotion after spinal cord injury. J Neurosci 25, 4694−4705 (2005).
10 Glaser, T., Perez-Bouza, A., Klein, K., and Brustle, O. Generation of purified oligodendrocyte progenitors from embryonic stem cells. Faseb J 19, 112−114 (2005).
11 Perez-Bouza, A., Glaser, T., and Brustle, O. ES cell-derived glial precursors contribute to remyelination in acutely demyelinated spinal cord lesions. Brain Pathol 15, 208−216 (2005).
12 Wang, S., Bates, J., Li, X., Schanz, S., Chandler-Militello, D., Levine, C., Maherali, N., Studer, L., Hochedlinger, K., Windrem, M., and Goldman, S. A. Human iPSC-derived oligodendrocyte progenitor cells can myelinate and rescue a mouse model of congenital hypomyelination. Cell Stem Cell 12, 252-264 (2013).
13 Piao, J., Major, T., Auyeung, G., Policarpio, E., Menon, J., Droms, L., Gutin, P., Uryu, K., Tchieu, J., Soulet, D., and Tabar, V. Human embryonic stem cell-derived oligodendrocyte progenitors remyelinate the brain and rescue behavioral deficits following radiation. Cell Stem Cell 16, 198−210 (2015).
14 Douvaras, P., Wang, J., Zimmer, M., Hanchuk, S., O'Bara, M. A., Sadiq, S., Sim, F. J., Goldman, J., and Fossati, V. Efficient generation of myelinating oligodendrocytes from primary progressive multiple sclerosis patients by induced pluripotent stem cells. Stem Cell Reports 3, 250−259 (2014).
15 Matalon, R., Michals, K., Sebesta, D., Deanching, M., Gashkoff, P., and Casanova, J. Aspartoacylase deficiency and N-acetylaspartic aciduria in patients with Canavan disease. Am J Med Genet 29, 463−471 (1988).
16 Leone, P., Janson, C. G., Bilaniuk, L., Wang, Z., Sorgi, F., Huang, L., Matalon, R., Kaul, R., Zeng, Z., Freese, A., McPhee, S. W., Mee, E., and During, M. J. Aspartoacylase gene transfer to the mammalian central nervous system with therapeutic implications for Canavan disease. Ann Neurol 48, 27−38 (2000).
17 Janson, C., McPhee, S., Bilaniuk, L., Haselgrove, J., Testaiuti, M., Freese, A., Wang, D. J., Shera, D., Hurh, P., Rupin, J., Saslow, E., Goldfarb, O., Goldberg, M., Larijani, G., Sharrar, W., Liouterman, L., Camp, A., Kolodny, E., Samulski, J., and Leone, P. Clinical protocol. Gene therapy of Canavan disease: AAV-2 vector for neurosurgical delivery of aspartoacylase gene (ASPA) to the human brain. Hum Gene Ther 13, 1391−1412 (2002).
18 Matalon, R., Surendran, S., Rady, P. L., Quast, M. J., Campbell, G. A., Matalon, K. M., Tyring, S. K., Wei, J., Peden, C. S., Ezell, E. L., Muzyczka, N., and Mandel, R. J. Adeno-associated virus-mediated aspartoacylase gene transfer to the brain of knockout mouse for canavan disease. Mol Ther 7, 580-587 (2003).
19 McPhee, S. W., Francis, J., Janson, C. G., Serikawa, T., Hyland, K., Ong, E. O., Raghavan, S. S., Freese, A., and Leone, P. Effects of AAV-2-mediated aspartoacylase gene transfer in the tremor rat model of Canavan disease. Brain Res Mol Brain Res 135, 112−121 (2005).
20 McPhee, S. W., Janson, C. G., Li, C., Samulski, R. J., Camp, A. S., Francis, J., Shera, D., Lioutermann, L., Feely, M., Freese, A., and Leone, P. Immune responses to AAV in a phase I study for Canavan disease. J Gene Med 8, 577−588 (2006).
21 Leone, P., Shera, D., McPhee, S. W., Francis, J. S., Kolodny, E. H., Bilaniuk, L. T., Wang, D. J., Assadi, M., Goldfarb, O., Goldman, H. W., Freese, A., Young, D., During, M. J., Samulski, R. J., and Janson, C. G. Long-term follow-up after gene therapy for canavan disease. Sci Transl Med 4, 165ra163 (2012).
22 Ahmed, S. S., Li, H., Cao, C., Sikoglu, E. M., Denninger, A. R., Su, Q., Eaton, S., Liso Navarro, A. A., Xie, J., Szucs, S., Zhang, H., Moore, C., Kirschner, D. A., Seyfried, T. N., Flotte, T. R., Matalon, R., and Gao, G. A single intravenous rAAV injection as late as P20 achieves efficacious and sustained CNS Gene therapy in Canavan mice. Mol Ther 21, 2136−2147 (2013).
23 Zano, S., Malik, R., Szucs, S., Matalon, R., and Viola, R. E. Modification of aspartoacylase for potential use in enzyme replacement therapy for the treatment of Canavan disease. Mol Genet Metab 102, 176−180 (2011).
24 Madhavarao, C. N., Arun, P., Anikster, Y., Mog, S. R., Staretz-Chacham, O., Moffett, J. R., Grunberg, N. E., Gahl, W. A., and Namboodiri, A. M. Glyceryl triacetate for Canavan disease: a low-dose trial in infants and evaluation of a higher dose for toxicity in the tremor rat model. J Inherit Metab Dis 32, 640-650 (2009).
25 Assadi, M., Janson, C., Wang, D. J., Goldfarb, O., Suri, N., Bilaniuk, L., and Leone, P. Lithium citrate reduces excessive intra-cerebral N-acetyl aspartate in Canavan disease. Eur J Paediatr Neurol 14, 354−359 (2010).
26 Francis, J. S., Markov, V., and Leone, P. Dietary triheptanoin rescues oligodendrocyte loss, dysmyelination and motor function in the nur7 mouse model of Canavan disease. J Inherit Metab Dis 37, 369−381 (2014).
27 Arun, P., Madhavarao, C. N., Moffett, J. R., Hamilton, K., Grunberg, N. E., Ariyannur, P. S., Gahl, W. A., Anikster, Y., Mog, S., Hallows, W. C., Denu, J. M., and Namboodiri, A. M. Metabolic acetate therapy improves phenotype in the tremor rat model of Canavan disease. J Inherit Metab Dis 33, 195−210 (2010).
28 Matalon, R. and Michals-Matalon, K. Recent advances in Canavan disease. Adv Pediatr 46, 493−506 (1999).
29 Kaul, R., Gao, G. P., Balamurugan, K., and Matalon, R. Cloning of the human aspartoacylase cDNA and a common missense mutation in Canavan disease. Nat Genet 5, 118−123 (1993).
30 Kaul, R., Gao, G. P., Aloya, M., Balamurugan, K., Petrosky, A., Michals, K., and Matalon, R. Canavan disease: mutations among Jewish and non-Jewish patients. Am J Hum Genet 55, 34−41 (1994).
31 Okita, K., Ichisaka, T., and Yamanaka, S. Generation of germline-competent induced pluripotent stem cells. Nature 448, 313−317 (2007).
32 Okita, K., Matsumura, Y., Sato, Y., Okada, A., Morizane, A., Okamoto, S., Hong, H., Nakagawa, M., Tanabe, K., Tezuka, K., Shibata, T., Kunisada, T., Takahashi, M., Takahashi, J., Saji, H., and Yamanaka, S. A more efficient method to generate integration-free human iPS cells. Nat Methods 8, 409−412 (2011).
33 Liu, G. H., Qu, J., Suzuki, K., Nivet, E., Li, M., Montserrat, N., Yi, F., Xu, X., Ruiz, S., Zhang, W., Wagner, U., Kim, A., Ren, B., Li, Y., Goebl, A., Kim, J., Soligalla, R. D., Dubova, I., Thompson, J., Yates, J., 3rd, Esteban, C. R., Sancho-Martinez, I., and Izpisua Belmonte, J. C. Progressive degeneration of human neural stem cells caused by pathogenic LRRK2. Nature 491, 603−607 (2012).
34 Traka, M., Wollmann, R. L., Cerda, S. R., Dugas, J., Barres, B. A., and Popko, B. Nur7 is a nonsense mutation in the mouse aspartoacylase gene that causes spongy degeneration of the CNS. J Neurosci 28, 11537−11549 (2008).

35 Matalon, R., Rady, P. L., Platt, K. A., Skinner, H. B., Quast, M. J., Campbell, 35 G. A., Matalon, K., Ceci, J. D., Tyring, S. K., Nehls, M., Surendran, S., Wei, J., Ezell, E. L., and Szucs, S. Knock-out mouse for Canavan disease: a model for gene transfer to the central nervous system. J Gene Med 2, 165−175 (2000).
36 Mersmann, N., Tkachev, D., Jelinek, R., Roth, P. T., Mobius, W., Ruhwedel, T., Ruhle, S., Weber-Fahr, W., Sartorius, A., and Klugmann, M.
Aspartoacylase-lacZ knockin mice: an engineered model of Canavan disease. PLOS One 6, e20336 (2011).
37 Crawley, J. N. Behavioral phenotyping of transgenic and knockout mice: experimental design and evaluation of general health, sensory functions, motor abilities, and specific behavioral tests. Brain Res 835, 18−26 (1999).
38 Janson, C. G., Assadi, M., Francis, J., Bilaniuk, L., Shera, D., and Leone, P. Lithium citrate for Canavan disease. Pediatr Neurol 33, 235−243 (2005).
39 Baslow, M. H., Kitada, K., Suckow, R. F., Hungund, B. L., and Serikawa, T. The effects of lithium chloride and other substances on levels of brain N-acetyl-L-aspartic acid in Canavan disease-like rats. Neurochem Res 27, 403-406 (2002).
40 Solsona, M. D., Fernandez, L. L., Boquet, E. M., and Andres, J. L. Lithium citrate as treatment of Canavan disease. Clin Neuropharmacol 35, 150−151 (2012).
41 Surendran, S., Shihabuddin, L. S., Clarke, J., Taksir, T. V., Stewart, G. R., Parsons, G., Yang, W., Tyring, S. K., Michals-Matalon, K., and Matalon, R. Mouse neural progenitor cells differentiate into oligodendrocytes in the brain of a knockout mouse model of Canavan disease. Brain Res Dev Brain Res 153, 19−27 (2004).
42 Uchida, N., Chen, K., Dohse, M., Hansen, K. D., Dean, J., Buser, J. R., Riddle, A., Beardsley, D. J., Wan, Y., Gong, X., Nguyen, T., Cummings, B. J., Anderson, A. J., Tamaki, S. J., Tsukamoto, A., Weissman, I. L., Matsumoto, S. G., Sherman, L. S., Kroenke, C. D., and Back, S. A. Human neural stem cells induce functional myelination in mice with severe dysmyelination. Sci Transl Med 4, 155ra136 (2012).
43 Madhavarao, C. N., Hammer, J. A., Quarles, R. H., and Namboodiri, M. A. A radiometric assay for aspartoacylase activity in cultured oligodendrocytes. Anal Biochem 308, 314−319 (2002).
44 Le Belle, J. E., Harris, N. G., Williams, S. R., and Bhakoo, K. K. A comparison of cell and tissue extraction techniques using high-resolution 1H-NMR spectroscopy. NMR Biomed 15, 37−44 (2002).
45 West, J. B., Fu, Z., Deerinck, T. J., Mackey, M. R., Obayashi, J. T., and Ellisman, M. H. Structure-function studies of blood and air capillaries in chicken lung using 3D electron microscopy. Respir Physiol Neurobiol 170, 202−209 (2010).
46 Clarner, T., Wieczorek, N., Krauspe, B., Jansen, K., Beyer, C., and Kipp, M. Astroglial redistribution of aquaporin 4 during spongy degeneration in a Canavan disease mouse model. J Mol Neurosci 53, 22−30 (2014).

1. A method for treating Canavan disease in a subject, comprising:
reprogramming or converting somatic cells isolated from the subject into induced pluripotent stem cells (iPSCs);
obtaining ASPA neural progenitor cells (NPCs) expressing wild type or functional ASPA;
wherein at least 90% of the ASPA NPCs are CD133 positive and SSEA4 negative; and
transplanting the ASPA NPCs into the brain of the subject, wherein the ASPA NPCs are obtained by:
differentiating the iPSCs into NPCs and introducing wild type ASPA gene into the NPCs by (i) transducing the NPCs with a lentivirus vector comprising the wild type ASPA gene, or (ii) correcting mutated ASPA gene of the NPCs by CRISPR/Cas 9 or TALEN-based gene editing.

2. The method of claim 1, wherein the reprogramming is carried out in the presence of one or more reprogramming factors comprising OCT4, SOX2, KLF4, LIN28 and MYC.

3. (canceled)

4. The method of claim 1, wherein the ASPA gene of the reprogrammed iPSCs comprises one or more mutations.

5. The method of claim 4, wherein the ASPA gene mutation is a heterozygous mutation.

6. The method of claim 4, wherein the ASPA gene mutation is a homozygous mutation.

7. The method of claim 4, wherein the ASPA gene mutation is 527G>A, 914C>A, or 854A>C.

8. The method of claim 1, wherein the somatic cells are fibroblasts, blood cells, urinary cells, adipocytes, keratinocytes, or dental pulp cells.

9-13. (canceled)

14. A method of producing ASPA neural precursor cells, comprising:
reprogramming or converting somatic cells isolated from a subject suffering from Canavan disease into induced pluripotent stem cells (iPSCs); and
obtaining ASPA neural precursor cells expressing wild type or functional ASPA by conducting a genetical correction of the iPSCs before or after differentiating the iPSCs into the neural precursor cells,
wherein the ASPA neural precursor cells are obtained by:
introducing wild type or functional ASPA gene in the iPSCs to obtain genetically corrected iPSCs which express wild type or functional ASPA,
wherein at least 90% of the ASPA NPCs are CD133 positive and SSEA4 negative; and
differentiating the genetically corrected iPSCs into neural precursor cells or differentiating the iPSCs into neural precursor cells; and introducing wild type or function ASPA gene into the neural precursor cells.

15. The method of claim 14, wherein the reprogramming is carried out, in the presence of one or more reprogramming factors comprising OCT4, SOX2, KLF4, LIN28 and MYC.

16. The method of claim 14, wherein the reprogramming is carried out via episomal reprogramming or viral transduction.

17. The method of claim 14, wherein the somatic cells are fibroblasts, blood cells, urinary cells, adipocytes, keratinocytes, dental pulp cells, or other easily accessible somatic cells.

18. The method of claim 14, wherein the wild type or functional ASPA gene is introduced by transducing the reprogrammed or converted iPSCs with a vector comprising the wild type or functional ASPA gene or by correcting the ASPA gene mutation using gene editing technology.

19. The method of claim 18, wherein the vector is lentivirus.

20. The method of claim 14, wherein the neural precursor cells include NPCs, glial progenitor cells and oligodendroglial progenitor cells.

21-27. (canceled)

28. The method of claim 1, wherein the NPCs are differentiated into oligodendrocyte progenitor cells (OPCs) and oligodendrocytes.

29. A method for treating Canavan disease in a subject, comprising:
(a) the population comprising NPCs that are:
(i) derived from differentiated iPSCs that are in turn derived from reprogrammed or converted somatic cells isolated from the subject, and
(ii) genetically corrected to express a wild type or functional ASPA gene by:
transducing the NPCs with a lentivirus vector comprising the wild type or functional ASPA gene, or
correcting mutated ASPA gene of the NPCs by CRISPR/Cas 9 or TALEN-based gene editing;
(iii) at least 90% CD133 positive and SSEA4 negative; and
(b) transplanting, into the brain of the subject, the population of ASPA NPCs.

30. The method of claim 29, wherein the somatic cells are fibroblasts, blood cells, urinary cells, adipocytes, keratinocytes, or dental pulp cells.
